(19) United States
(12) Patent Application Publication
(10) Pub. No.: US 2021/0055259 A1
(43) Pub. Date: Feb. 25, 2021

(54) Electrochemical Detection Method Based on Tracers Labeling

(71) Applicants: BEIJING SAVANT BIOTECHNOLOGY CO., LTD., Beijing (CN); TIANJIN SAVANT BIOTECHNOLOGY CO., LTD., Tianjin (CN)

(72) Inventor: Si LIN, Beijing (CN)

(73) Assignees: BEIJING SAVANT BIOTECHNOLOGY CO., LTD., Beijing (CN); TIANJIN SAVANT BIOTECHNOLOGY CO., LTD., Tianjin (CN)

(21) Appl. No.: 16/961,701

(22) PCT Filed: Dec. 26, 2018
(86) PCT No.: PCT/CN2018/123835

§ 371 Date: Jul. 13, 2020

(30) Foreign Application Priority Data
Jun. 29, 2018 (CN) 201810699669.2
Sep. 6, 2018 (CN) 201811037500.7

(51) Int. Cl. G01N 27/48; G01N 33/543

(52) U.S. Cl. CPC G01N 27/48; G01N 33/54326; G01N 33/54306

(57) ABSTRACT

An electrochemical detection method based on tracers labeling uses the technical solution involving three similar methods, and method I as a basic method comprising the steps of: (1) labeling an antibody of the analyte with a tracer; (2) labeling another antibody of the analyte with one of a pair of substances having specific affinity; (3) labeling a nano-microsphere with the other of a pair of substances having specific affinity; (4) performing electrochemical detection process. In the present invention, by utilizing the electrochemical properties of the tracers and the high sensitivity of the detection of tracers by the electrochemical method, the tracers-labeled immune complex is collected through the nano-microsphere, then the nano-microsphere with the immune complex are enriched onto the surface of an electrode through separation, which greatly improves the detection sensitivity; the method is high in stability, good in repeatability, accurate and reliable in result.

### CROSS REFERENCE TO THE RELATED APPLICATIONS

This application the national phase entry of International Application No. PCT/CN2018/123835, filed on Dec. 26, 2018, which is based on and claims priority to Chinese Patent Application No. 201810699669.2, filed Jun. 29, 2018, and Chinese Patent Application No. 201811037500.7, filed Sep. 6, 2018, the entire contents of which are incorporated herein by reference.

### TECHNICAL FIELD

The invention belongs to the field of medical diagnostic, and particularly relates to an electrochemical detection method based on tracers labeling.

### BACKGROUND

Electrochemical detection technology is an emerging sensitive analysis and detection technology in recent years, which is an analysis and detection technology with different electrical signals as excitation and detection signals. Electrochemical detection technology has been favored for its advantages of simple operation, high sensitivity and high detection speed. It has been widely studied and applied in the fields of life science, biological science, clinical analysis, environmental monitoring and surface science.

The electrochemical sensor is a sensor component that combines electrochemical analysis and sensing technology to achieve economical, efficient, practical, fast, sensitive and accurate detection and analysis. And the detection principle is based on the influence of the measured substance on the electrochemical signal of the electrode system, thereby realizing the quantitative analysis of the measured substance.

In the current study, immunoassays using metal ions as tracers are usually performed by mass spectrometry immunoassay, and electrochemical methods are rarely used. In addition, in the current electrochemical detection method, generally, the characteristic peak height of the characteristic substance in the voltamogram is used to establish a relationship with the concentration of the analyte. However, the voltammogram is susceptible to an overall shift in the influence of the detection environment, and the value of the peak height is also affected to varying degrees, resulting in reduced accuracy of detection.

### SUMMARY

The problem to be solved by the invention is to provide an electrochemical detection method based on tracers labeling, which has the advantages of improved detection sensitivity, high stability for method, good repeatability, and fast, accurate and reliable result.

In order to solve the technical problem, the present invention adopts the following technical solutions:

An electrochemical detection method based on tracers labeling, comprising the steps of:

(1) preparing a tracer-labeled immune complex in a reaction cell;

(2) enriching the tracer-labeled immune complex on the surface of a working electrode through a separation method, removing residual liquid in the reaction cell, and filling an electrolyte;

(3) connecting the electrode to an electrochemical workstation, measuring a voltammogram of the tracer by a voltammetric method, picking out a characteristic peak of the tracer in the measured voltammogram, calculating a half-peak area, fitting a curve between the half-peak area and the concentration of the analyte by using a regression equation to obtain a standard curve, and calculating the content of the analyte by the standard curve.

Further, wherein in step (3), the characteristic peak is located within a range of ±100 mV of the theoretical characteristic peak of the metal ions; and the regression equation is a Log-Log or a Log-Logit regression equation.

Further, wherein the tracer-labeled immune complex is prepared by the steps of:

(1) labeling an antibody of the analyte with a tracer;

(2) labeling another antibody of the analyte with one of a pair of substances having specific affinity;

(3) labeling a nano-microsphere with the other of a pair of substances having specific affinity;

(4) adding a sample containing the analyte, the tracer-labeled antibody of the analyte and another antibody of the analyte labeled with one of a pair of substances having specific affinity into a reaction cell, performing an incubation reaction for 3-90 min, and sequentially adding the nano-microsphere labeled with the other of a pair of substances having specific affinity on the surface to form a tracer-labeled immune complex;

or,

(1) labeling an antibody of the analyte with a tracer;

(2) labeling a complete antigen of the analyte with one of a pair of substances having specific affinity;

(3) labeling a nano-microsphere with the other of a pair of substances having specific affinity;

(4) adding a sample containing the analyte, the tracer-labeled antibody of the analyte and the complete antigen of the analyte labeled with one of a pair of substances having specific affinity into a reaction cell, performing an incubation reaction for 3-90 min, and sequentially adding the nano-microsphere labeled with the other of a pair of substances having specific affinity on the surface to form a tracer-labeled immune complex;

or,

(1) labeling a complete antigen of the analyte with a tracer;

(2) labeling an antibody of the analyte with one of a pair of substances having specific affinity;

(3) labeling a nano-microsphere with the other of a pair of substances having specific affinity;

(4) adding a sample containing the analyte, the tracer-labeled complete antigen of the analyte and the antibody of the analyte labeled with one of a pair of substances having specific affinity into a reaction cell, performing an incubation reaction for 3-90 min, and sequentially adding the nano-microsphere labeled with the other of a pair of substances having specific affinity on the surface to form a tracer-labeled immune complex.

Further, wherein the tracers are metal ion materials; the electrochemical detection is performed using a three-electrode system or a two-electrode system.

Further, wherein the metal ion materials are microspheres containing metal ions on the surface or inside; the metal ion is selected from the group consisting of Cd²⁺, Cu²⁺, Zn²⁺, Mn²⁺, Pb²⁺, Ag⁺, Li⁺, Hg²⁺, Co²⁺, Cr³⁺, Ni²⁺, Au³⁺, and Ba²⁺ ions; the microsphere is selected from the group consisting of polystyrene microsphere, polytetrafluoroethylene microsphere, titanium dioxide microsphere, manganese dioxide microsphere, zirconium dioxide microsphere, organic silicon microsphere, polyamide microsphere, polyacrylic acid microsphere, chitosan microsphere, polyaniline microsphere, polyvinyl chloride microsphere, cobalt microsphere, nickel microsphere, platinum microsphere, gold microsphere, silver microsphere, palladium microsphere, silicon dioxide microsphere and magnetic microsphere.

Further, wherein the nano-microsphere is selected from the group consisting of polystyrene microsphere, polytetrafluoroethylene microsphere, silica microsphere, titanium dioxide microsphere, organic silicon microsphere, polyamide microsphere, polyacrylic acid microsphere, chitosan microsphere, polyaniline microsphere, polyvinyl chloride microsphere and magnetic microsphere.

Further, wherein the separation method adopts a centrifugal separation, electric field or capillary action; or when the nano-microsphere is made of magnetic microsphere, the separation method adopts a magnetic separation; the magnetic microsphere is magnetic Fe₃O₄, γ-Fe₂O₃, Pt, Ni or Co microsphere, or core/shell or doped microsphere formed by combining magnetic Fe₃O₄, γ-Fe₂O₃, Pt, Ni or Co with inorganic matters or organic matters.

Further, wherein the metal ion material has a particle size of 1-500 nm and the nano-microsphere has a particle size of 50 nm-5 μm.

Further, wherein in step (3), the regression equation is a four-parameter regression equation.

Still further, wherein the tracer-labeled immune complex is prepared by the steps of:

Step 1, labeling an antibody or antigen of the analyte with a tracer;

Step 2, labeling another antibody of the analyte with one of a pair of substances having specific affinity;

Step 3, labeling a magnetic microsphere with the other of a pair of substances having specific affinity;

Step 4, adding the analyte, the tracer-labeled antibody or antigen and another antibody labeled with one of a pair of substances having specific affinity into a detection cell, performing an incubation reaction, and sequentially adding the magnetic microsphere labeled with the other of a pair of substances having specific affinity to form a tracer-labeled immune complex;

or,

Step 1, labeling an antibody or antigen of the analyte with a tracer;

Step 2, labeling another antibody of the analyte with magnetic microsphere;

Step 3, adding the analyte, the tracer-labeled antibody or antigen of the analyte and another magnetic microsphere-labeled antibody of the analyte into a detection cell, and performing an incubation reaction to form a tracer-labeled immune complex;

or,

Step 1, labeling an antibody or antigen of the analyte with one of a pair of substances having specific affinity;

Step 2, labeling another antibody of the analyte with a magnetic microsphere;

Step 3, labeling the other of a pair of substances having specific affinity with a tracer;

Step 4, adding the analyte, an antibody or antigen of the analyte labeled with one of a pair of substances having specific affinity, and another magnetic microsphere-labeled antibody of the analyte into a detection cell, performing an incubation reaction, and sequentially adding the other of a pair of substances having specific affinity labeled with the tracer to form a tracer-labeled immune complex.

Still further, the tracer-labeled immune complex comprises a tracer-labeled antibody or antigen, another magnetic microsphere-labeled antibody of the analyte, and the analyte.

Still further, wherein the tracer-labeled antibody or antigen is linked by a pair of substances having specific affinity.

Further, the pair of substances having specific affinity is biotin and streptavidin, biotin and avidin, fluorescein and anti-fluorescein, or antibody and secondary antibody specifically binding the antibody;

Still further, another magnetic microsphere-labeled antibody of the analyte is linked by a pair of substances having specific affinity.

Further, the pair of substances having specific affinity is biotin and streptavidin, biotin and avidin, fluorescein and anti-fluorescein, or antibody and secondary antibody specifically binding the antibody.

Further, the tracer is a metal oxide material; the detection is performed using a four electrode system.

Further, the metal oxide is copper oxide.

Further, the copper oxide is selected from the group consisting of 1) bare copper oxide nanoparticles; or 2) one of copper oxides with surface coated by a layer of silicon dioxide, titanium dioxide, carbonate, silicate, phosphate, silicon carbide, graphite and silicon nitride; or 3) one of copper oxides with surface coated by a layer of organic silicon, polystyrene, polytetrafluoroethylene, polyamide, polyethylene, polyvinyl chloride, polyvinyl fluoride, polyacrylonitrile, polyamide, polyimide, polyaniline, polypyrrole, polyacrylic acid, chitosan, polylactic acid, epoxy resin, phenolic resin, polyacetylene, polyester, β-cyclodextrin polymer, vitamin and melamine; the antibody or antigen is the antibody or antigen of the analyte.

Further, the four-electrode system adopts a screen printing electrode consisting of a working electrode, an internal control electrode, a counter electrode and a reference electrode respectively, the screen printing electrode is inserted into a detection cell, and a magnet is arranged under the corresponding working electrode of the screen printing electrode in the detection cell.

Further, the working electrode is a copper electrode, a carbon electrode, a glassy carbon electrode, a gold microelectrode, a graphite electrode, a silver electrode, a lead electrode, or an electrode doped with graphene or fullerene in the above electrode, or an electrode modified, coated, doped or attached to graphene or fullerene on the surface of the above electrode; the internal control electrode is a copper electrode, a carbon electrode, a glassy carbon electrode, a gold microelectrode, a graphite electrode, a silver electrode, a lead electrode, or an electrode doped with graphene or fullerene among the above electrode, or an electrode modified, coated, doped or attached to graphene or fullerene on the surface of the above electrode; the counter electrode is a platinum wire electrode or a carbon electrode; and the reference electrode is a calomel electrode or an Ag/AgCl electrode.

The invention also provides an electrochemical detection method based on metal ions labeling, comprising the steps of:

(1) labeling an antibody of the analyte with a metal ion material;

(2) labeling another antibody of the analyte with one of a pair of substances having specific affinity;

(3) labeling a nano-microsphere with the other of a pair of substances having specific affinity;

(4) performing a detection process:

1) adding a sample containing the analyte, the metal ion material-labeled antibody of the analyte and another antibody of the analyte labeled with a pair of substances having specific affinity into a reaction cell, and performing an incubation reaction for 3-90 min to form an immune complex labeled with the metal ion material at one end, and labeled with one of the pair of substances having specific affinity at the other end;

2) then adding the nano-microsphere with surfaces-labeled the other of a pair of substances having specific affinity into the mixture reacted from step 1), wherein one of the pair of substances having specific affinity at one end of the immune complex in step 1) specifically binds to the other of a pair of substances having specific affinity labeled on the surface of the nano-microsphere, forming nano-microsphere with surface-bound the immune complex in step 1);

3) fixing the nano-microsphere in step 2) with surface-bound the immune complex in step 1) to the surface of the working electrode in a separation method, then removing residual liquid in the reaction cell, and filling electrolyte;

4) performing measurement by using a three-electrode system or a two-electrode system, connecting electrodes to an electrochemical workstation, measuring a voltammogram of metal ions on an immune complex on the surface of nano-microsphere by using an electrochemical detection method, then picking out an actual characteristic peak within the range of +/−100 mV of a theoretical characteristic peak of the metal ions in the measured voltammogram, calculating a half-peak area, fitting a curve between the half-peak area and the concentration of the analyte by using a Log-Log regression equation to obtain a standard curve, and calculating the content of the analyte by the standard curve.

The invention further provides an electrochemical detection method based on metal ions labeling, comprising the steps of:

(1) labeling an antibody of the analyte with a metal ion material;

(2) labeling a complete antigen of the analyte with one of a pair of substances having specific affinity;

(3) labeling a nano-microsphere with the other of a pair of substances having specific affinity;

(4) performing a detection process:

1) adding a sample containing the analyte, a metal ion material-labeled antibody of the analyte, and the complete antigen of the analyte labeled with one of the pair of substances having specific affinity into a reaction cell, wherein the analyte competes with the complete antigen of the analyte labeled with one of a pair of substances having specific affinity for an immunoreaction with the metal ion-labeled antibody of the analyte, and performing an incubation reaction for 3-90 min to form a first immune complex labeled with the metal ion material at one end and with one of the pair of substances having specific affinity at the other end; and to form a second immune complex labeled with the metal ion material at one end;

2) then adding the nano-microsphere with surface-labeled the other of a pair of substances having specific affinity to the mixture reacted from step 1), wherein one of the pair of substances having specific affinity at one end of the first immune complex in step 1) specifically binds to the other of a pair of substances having specific affinity labeled on the surface of nano-microsphere, forming a nano-microsphere with surfaces-bound the first immune complex, and the second immune complex does not participate in reaction;

3) fixing the nano-microsphere with surface-bound the first immune complex in step 2) to the surface of the working electrode through a separation method, then removing the residual liquid in the reaction cell, and filling the electrolyte;

4) performing measurement by using a three-electrode system or a two-electrode system, connecting electrodes to an electrochemical workstation, measuring a voltammogram of metal ions on an immune complex on the surface of the nano-microsphere by using an electrochemical detection method, then picking out an actual characteristic peak within the range of +/−100 mV of a theoretical characteristic peak of the metal ions in the measured voltammogram, calculating a half-peak area, fitting a curve between the half-peak area and the concentration of the analyte by using a Log-Logit regression equation to obtain a standard curve, and calculating the content of the analyte by the standard curve.

The invention further provides an electrochemical detection method based on metal ions labeling, comprising the steps of:

(1) labeling a complete antigen of the analyte with a metal ion material;

(2) labeling an antibody of the analyte with one of a pair of substances having specific affinity;

(3) labeling a nano-microsphere with the other of a pair of substances having specific affinity;

(4) performing a detection process:

1) adding a sample containing the analyte, a metal ion material-labeled complete antigen of the analyte, and the antibody of the analyte labeled with one of the pair of substances having specific affinity into a reaction cell, wherein the analyte competes with the metal ion-labeled complete antigen of the analyte for an immunoreaction with the antibody of the analyte labeled with one of a pair of substances having specific affinity, and performing an incubation reaction for 3-90 min to form a first immune complex labeled with the metal ion material at one end and with one of the pair of substances having specific affinity at the other end; and to form a second immune complex labeled with one of the pair of substances having specific affinity at one end;

2) then adding the nano-microsphere with surface-labeled the other of a pair of substances having specific affinity to the mixture reacted from step 1), wherein the ones of a pair of substances having specific affinity at one end of the first immune complex and the second immune complex in step 1) specifically bind to the other of a pair of substances having specific affinity labeled on the surface of nano-microsphere respectively, forming the nano-microsphere with surface-bound the first immune complex and the second immune complex;

3) fixing the nano-microsphere with surface-bound the first immune complex and the second immune complex in step 2) to the surface of the working electrode in a separation method, then removing the residual liquid in the reaction cell, and filling the electrolyte;

4) performing measurement by using a three-electrode system or a two-electrode system, connecting the electrodes to an electrochemical workstation, measuring a voltammogram of metal ions on the immune complex on the surface of the nano microsphere by using an electrochemical detection method, then picking out an actual characteristic peak within the range of +/−100 mV of a theoretical characteristic peak of the metal ions in the measured voltammogram, calculating a half-peak area, fitting a curve between the half-peak area and the concentration of the analyte by using a Log-Logit regression equation to obtain a standard curve, and calculating the content of the analyte by the standard curve.

Further, the metal ions materials are microspheres containing metal ions on the surface or inside; the metal ion is selected from the group consisting of Cd²⁺, Cu²⁺, Zn²⁺, Mn²⁺, Pb²⁺, Ag⁺, Li⁺, Hg²⁺, Co²⁺, Cr³⁺, Ni²⁺, Au³⁺, and Ba²⁺ ions; the microsphere is selected from the group consisting of polystyrene microsphere, polytetrafluoroethylene microsphere, titanium dioxide microsphere, manganese dioxide microsphere zirconium dioxide microsphere, organic silicon microsphere, polyamide microsphere, polyacrylic acid microsphere, chitosan microsphere, polyaniline microsphere, polyvinyl chloride microsphere, cobalt microsphere, nickel microsphere, platinum microsphere, gold microsphere, silver microsphere, palladium microsphere, silicon dioxide microsphere and magnetic microsphere.

Further, the nano-microsphere is selected from the group consisting of polystyrene microsphere, polytetrafluoroethylene microsphere, silica microsphere, titanium dioxide microsphere, organic silicon microsphere, polyamide microsphere, polyacrylic acid microsphere, chitosan microsphere, polyaniline microsphere, polyvinyl chloride microsphere and magnetic microsphere.

Further, the separation method adopts a centrifugal separation, electric field or capillary action; or when the nano microsphere is made of magnetic microsphere, the separation method adopts a magnetic separation; the magnetic microsphere is selected from the group consisting of magnetic Fe₃O₄, γ-Fe₂O₃, Pt, Ni or Co microsphere, or core/shell or doped microsphere formed by combining magnetic Fe₃O₄, γ-Fe₂O₃, Pt, Ni or Co with inorganic matters or organic matters.

Further, the metal ion material has a particle size of 1-500 nm and the nano-microsphere has a particle size of 50 nm-5 μm; the pair of substances having specific affinity is biotin and streptavidin, biotin and avidin, fluorescein and antifluorescein, and antibody and secondary antibody specifically binding the antibody.

Further, in step 3), after removing the remaining liquid, the nano-microsphere with surface-bound the first immune complex and the second immune complex may be washed 2-3 times with a PB buffer and refilled with electrolyte.

Further, the three-electrode system comprises a working electrode, a counter electrode and a reference electrode; the two-electrode system comprises a working electrode and a counter electrode; the working electrode is a copper electrode, a carbon electrode, a glassy carbon electrode, a gold microelectrode, or an electrode doped with graphene in the electrode or an electrode coated with graphene on the surface of the electrode; the counter electrode is a platinum wire electrode; the reference electrode is a calomel electrode or an Ag/AgCl electrode; the electrochemical detection method is selected from the group consisting of cyclic voltammetry, differential voltammetry, differential pulse voltammetry, alternating current impedance spectroscopy, anodic stripping voltammetry and differential pulse anodic stripping voltammetry.

Further, the electrolyte is a 0.01 M-0.6 M phosphate buffer solution with pH value of 7.4; 0.01 M-0.6 M citric acid buffer solution with pH value of 3-7; 0.01 M-0.6 M acetic acid buffer solution with pH value of 2-7; a potassium ferricyanide electrolyte consisting of 0.1 mM-1 M K₃[Fe(CN)₆]/K₄[Fe(CN)] and 0.1 mM-1 M KCl.

The invention has the following beneficial effects:

1. According to the abovementioned technical solution, metal ions or oxides such as copper oxide are used as the labeling materials, the electrochemical characteristics of the metal ions or the oxides and the high sensitivity of the detection of tracers by the electrochemical method are utilized, and the detection sensitivity can be greatly improved; the method is high in stability, good in repeatability, accurate and reliable in result, the purposes of rapid and sensitive detection are achieved, and the application of the electrochemical detection method based on the metal ions or oxides labeling in the field of in-vitro diagnosis is expanded.

2. Compared with the traditional electrochemical detection method in which the antibody or antigen of the analyte directly coated on the surface of the electrode, in this invention, the nano-microsphere is adopted to collect the immune complex, and then the nano-microsphere with the immune complex is enriched on the surface of the electrode through separation, so that the experimental error is reduced, and the detection sensitivity is improved.

3. At present, for an electrochemical detection method, a characteristic peak height of a characteristic substance in a voltammogram is generally adopted to establish a relationship with the concentration of the analyte; according to this invention, the characteristic peak of the characteristic substance in the voltammogram is found out, then the half-peak area is calculated, the relation between the half-peak area and the concentration of the analyte is used for establishing a standard curve, the content of the analyte is calculated through the standard curve, and the content of the analyte can be reflected more accurately and stably.

4. According to the invention, a screen printing electrode is adopted, and the screen printing electrode adopts a four-electrode system and is respectively provided with a working electrode, an internal control electrode, a counter electrode and a reference electrode. The internal control electrode is used for calibrating a base line, avoiding fluctuation of electrochemical reaction and reducing detection error.

### BRIEF DESCRIPTION OF THE DRAWINGS

FIG. 1 is a schematic diagram showing the reaction process of step (1) and step (2) in the detection process of method I in Embodiment 1 of the present invention;

FIG. 2 is a schematic diagram showing the detection of step (3) and step (4) in the detection process of method I in Embodiment 1 of the present invention;

FIG. 3 is a schematic diagram showing the reaction process of step (1) and step (2) in the detection process of method II in Embodiment 2 of the present invention;

FIG. 4 is a schematic diagram showing the detection of step (3) and step (4) in the detection process of method II in Embodiment 2 of the present invention;

FIG. 5 is a schematic diagram showing the reaction process of step (1) and step (2) in the detection processes of method III in Embodiment 3 of the present invention;

FIG. 6 is a schematic diagram showing the detection of step (3) and step (4) in the detection process of method III in Embodiment 3 of the present invention;

FIG. 7 is a voltammogram of Cd²⁺ measured after detection of FT₃ calibrator of various concentrations in Embodiment 2 of the present invention, a-0 pg/mL, b-1.8 pg/mL, c-4.5 pg/mL, d-7.5 pg/mL, e-12 pg/mL, f-40 pg/mL;

FIG. 8 is a standard curve graph of AFP of method I in Embodiment 1 of the present invention;

FIG. 9 is a standard curve graph of FT₃ of method II in Embodiment 2 of the present invention;

FIG. 10 is a standard curve graph of FT₃ of method III in Embodiment 3 of the present invention;

FIG. 11 is a voltammogram of copper ions in Embodiment 4, wherein the concentration of the PCT calibrator is (a) S₀=0 ng/mL, (b) S₁=0.02 ng/mL, (c) S₂=1 ng/mL, (d) S₃=10 ng/mL, (e) S₄=25 ng/mL, (f) S₅=100 ng/mL;

FIG. 12 is a four-parameter Logistic curve fitting data diagram in Embodiment 4;

FIG. 13 is a standard curve graph of PCT in Embodiment 4;

FIG. 14 is a graph showing correlation of PCT determination results in Embodiment 4;

FIG. 15 is a voltammogram of copper ions in Embodiment 5, wherein the concentration of the FT₄ calibrator is S₀=0 pg/mL, (b) S₁=1 pg/mL, (c) S₂=3 pg/mL, (d) S₃=10 pg/mL, (e) S₄=30 pg/mL, (f) S₅=100 pg/mL;

FIG. 16 is a four-parameter Logistic curve fitting data diagram in Embodiment 5;

FIG. 17 is a standard curve graph of FT₄ in Embodiment 5;

FIG. 18 is a graph showing the correlation of FT₄ determination results in Embodiment 5;

FIG. 19 is a four-parameter Logistic curve fitting data diagram in Embodiment 6;

FIG. 20 is a standard curve graph of Fer in Embodiment 6;

FIG. 21 is a graph showing the correlation of Fer determination results in Embodiment 6;

FIG. 22 is a four-parameter Logistic curve fitting data diagram in Embodiment 7;

FIG. 23 is a standard curve graph of FT₃ in Embodiment 7;

FIG. 24 is a graph showing the correlation of FT₃ determination results in Embodiment 7;

FIG. 25 is a four-parameter Logistic curve fitting data diagram in Embodiment 8;

FIG. 26 is a standard curve graph of HE4 in Embodiment 8;

FIG. 27 is a graph showing correlation of HE4 determination results in Embodiment 8;

FIG. 28 is a four-parameter Logistic curve fitting data diagram in Embodiment 9;

FIG. 29 is a standard curve graph of 25-OH-D in Embodiment 9;

FIG. 30 is a graph showing the correlation of 25-OH-D determination results in Embodiment 9:

FIG. 31 is a comparison of the stability of polystyrene microsphere with surface-coated Cu²⁺ as a tracer in Embodiment 10 (1), (a) detection the antibody labeled with Cu²⁺-coated polystyrene microsphere, and (b) detection the antibody labeled with Cu²⁺-coated polystyrene microsphere after placing at 37° C. for 3 days;

FIG. 32 is a comparison of stability of CuO as a tracer in Embodiment 10 (2), a) detection the antibody labeled with CuO, and b) detection the CuO-labeled antibody after placing at 37° C. for 3 days;

FIG. 33 is a voltammogram of CuO as a tracer in Embodiment 11 (1);

FIG. 34 is a voltammogram of zinc oxide as a tracer in Embodiment 11 (2);

FIG. 35 is a voltammogram of CuO as a tracer before and after base calibration using an internal control electrode in Embodiment 12;

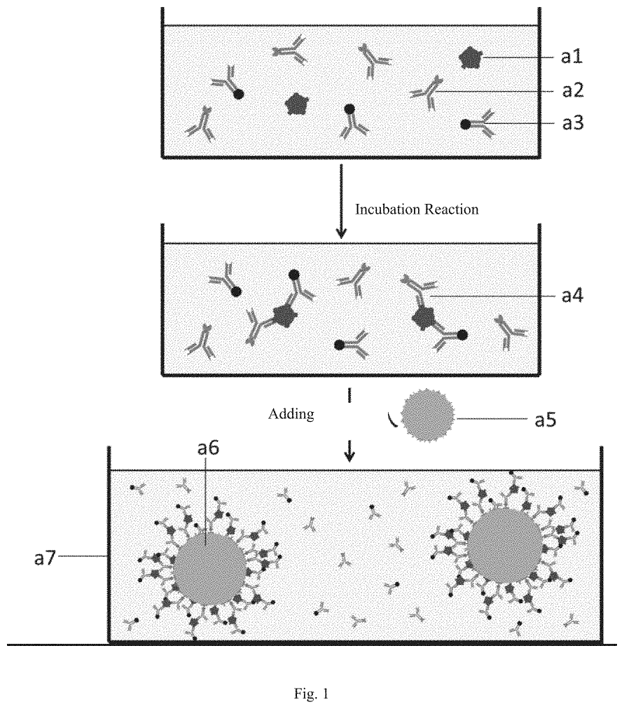

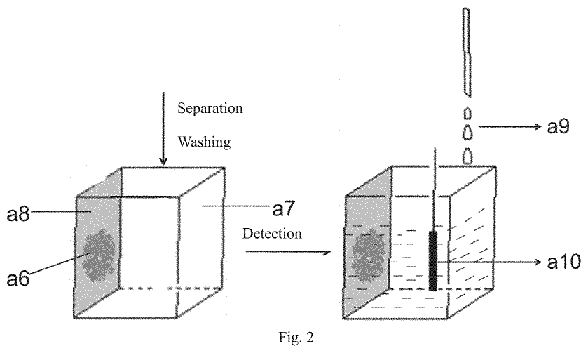

Wherein in FIGS. 1 and 2:

a1—AFP; a2—another mouse anti-human AFP monoclonal antibody labeled with biotin; a3—a mouse anti-human AFP monoclonal antibody labeled with copper ions materials; a4—an AFP immune complex labeled with copper ions materials at one end and biotin at the other end; a5—a magnetic microsphere labeled with streptavidin; a6—a magnetic microsphere with surface-bound AFP immune complex; a7—reaction cell; a8—working electrode; a9—electrolyte; a10—counter electrode;

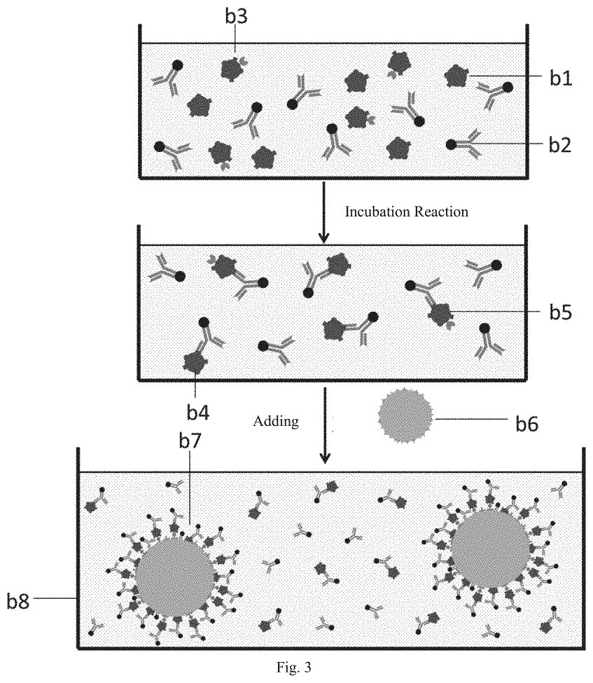

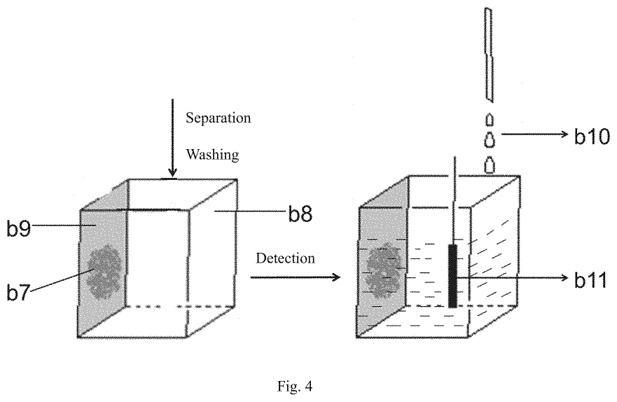

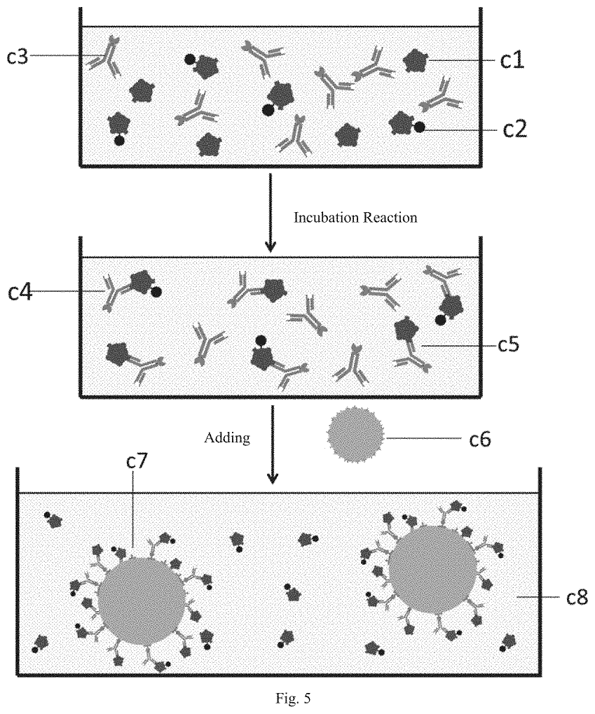

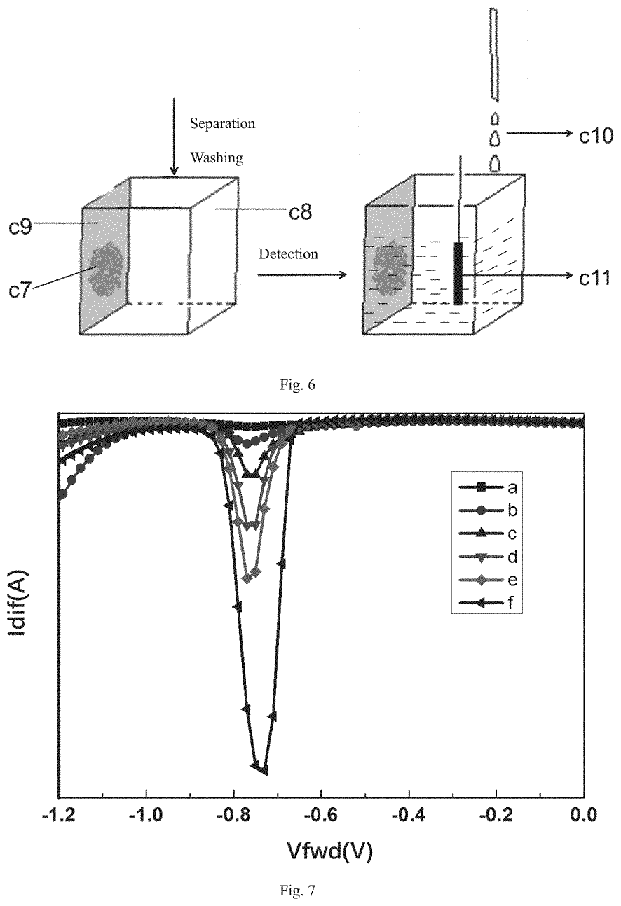

In FIGS. 3 and 4:
b1—FT₃; b2—a mouse anti-human FT₃ monoclonal antibody labeled with Embodiment 3:
Taking the detection of free triiodothyronine (FT₃) as an example
ions materials; b3—a FT₃ complete antigen labeled with biotin; b4—a second FT₃ immune complex labeled with cadmium ions at one end; b5—a first FT₃ immune complex labeled with cadmium ions at one end and biotin at the other end; b6—a magnetic nano-microsphere labeled with streptavidin; b7—a magnetic microsphere with surface-bound a first FT₃ immune complex; b8—reaction cell; b9—working electrode; b10—electrolyte; b11—counter electrode; In FIGS. 5 and 6:

c1—FT₃; c2—an FT₃ complete antigen labeled with cadmium ion materials; c3—a mouse anti-human FT₃ monoclonal antibody labeled with biotin; c4—a first FT₃ immune complex labeled with a cadmium ion material at one end and biotin at the other end; c5—a second FT₃ immune complex with biotin labeled at one end; c6—a magnetic nano-microsphere labeled with streptavidin; c7—a magnetic microsphere with surface-bound a first FT₃ immune complex and a second FT₃ immune complex; c8—reaction cell; c9—working electrode; c10—electrolyte; c11—counter electrode.

### DETAILED DESCRIPTION OF THE EMBODIMENTS

In order to further illustrate the technical means and functions adopted by the present invention for achieving the intended purposes of the present invention, the specific embodiments, structures, features and functions of the electrochemical detection method based on tracers labeling according to the present invention will be described in detail below with reference to the accompanying drawings and preferred examples.

Method I: as shown in FIGS. 1 and 2,

### Embodiment 1: Taking the Detection of Alpha-Fetoprotein (AFP) as an Example

1. Preparation of Copper Ions-Embedded Polystyrene Microspheres:

10 g of styrene monomer, 1 g of sodium lauryl sulfate, 100 mL of purified water, and 1 g of copper chloride powder were stirred and mixed uniformly, placed in a 75° C. incubator, and sequentially mixed uniformly by introducing nitrogen gas; then 1 g of potassium persulfate was dissolved in 5 mL of purified water, the mixture was added to the reaction solution at a rate of 0.1 mL/min, and reacted under nitrogen for 50 min; after finishing the reaction, 100 mL of ice water mixture was added to rapidly reduce the temperature of the reaction solution to below 40° C. The solution was centrifuged 3 times at 10,000 r/min for 30 min each time, redispersed and washed with 95% ethanol. Finally, the solution was re-dispersed with 100 mL of purified water; the redispersed material was placed in a 50° C. water bath for high-speed stirring, and a mixed acid of sulfuric acid and nitric acid (v/v=3:2) was added for reaction 2 h, and the reactant was centrifuged at 10,000 r/min to remove the supernatant, the precipitate was washed and redispersed with purified water to 100 ml, and this procedure was repeated 3 times. Finally, the redispersed material was transferred to a 75° C. incubator, 2 g of NaOH and 2 g of Na₂S₂O₄ were added. After stirring for reaction 4 h, the solution was centrifuged at 10,000 r/min to remove the supernatant, the precipitate was washed with and redispersed purified water to 100 mL, and then the procedure was repeated 3 times, and recorded as copper ion materials.

2. A Copper Ion Materials-Labeled Mouse Anti-Human AFP Monoclonal Antibody:

Firstly, a mouse anti-human AFP monoclonal antibody was diluted to 1 mg/mL with sodium carbonate buffer 1 and dialyzed against sodium carbonate buffer 1 after stirring for 4 h at room temperature (25° C.±5° C.) in the dark; 1 mL of mouse anti-human AFP monoclonal antibody solution was added into 5 mL of prepared copper ion materials, magnetically stirred at 25° C.±5° C. for 30 min in the dark, and then the mixed solution was transferred to a dialysis bag and dialyzed against a phosphate buffer at 4° C. overnight. Finally, the dialysate was taken out and stored at −20° C. by adding the same amount of glycerol.

3. Another Biotin-Labeled Mouse Anti-Human AFP Monoclonal Antibody:

Firstly, another mouse anti-human AFP monoclonal antibody was diluted to 1 mg/mL with sodium carbonate buffer 1 and dialyzed against sodium carbonate buffer 1 after stirring for 4 h at room temperature (25° C.±5° C.) in the dark; 6-aminocaproic acid-N-hydroxysuccinimide-biotin (BCNHS) was prepared into 1 mg/mL solution with N,N-dimethyl amide (DMF); 80 μL of the above DMF solution was added to 1 mL of another mouse anti-human AFP monoclonal antibody solution, the mixture was blended in a glass bottle, and stirred at room temperature (25° C.±5° C.) for 2 h in the dark; 9.6 μL of 1 mol/L ammonium chloride solution was added, and the mixture was stirred for 10 min at room temperature (25° C.±5° C.) in the dark; then the mixed solution was transferred to a dialysis bag and dialyzed against a phosphate buffer at 4° C. overnight. Finally, the dialyzed substance was taken out and stored at −20° C. by adding the same amount of glycerol.

4. Streptavidin-Labeled Magnetic Microspheres:

0.5 mL of 2.5 mg/mL magnetic microspheres (with a particle size of 800 nm, purchased from Tianjin Saierqun Technology Co., Ltd.) were washed twice with a phosphate buffer solution (pH=7.4) and then re-suspended in 10 mL of phosphate buffer solution, 4 mL of glutaraldehyde was sequentially added for shaking reaction for 10 h at room temperature, the reactant was washed with a large amount of purified water after finishing the reaction, and the magnetic microspheres were re-suspended in 10 mL of phosphate buffer solution; 0.5 mg of streptavidin was dissolved in 1 mL of phosphate buffer solution, then the mixture was added into the phosphate buffer solution of the magnetic microspheres for shaking 8 h at room temperature, the reactant was washed with the phosphate buffer solution, and dispersed in 10 mL of phosphate buffer solution for later use.

5. Detection Process:

(1) 10 μL of AFP a1-containing sample to be tested, 10 μL of a copper ion materials-labeled mouse anti-human AFP monoclonal antibody a3 and 10 μL of another biotin-labeled mouse anti-human AFP monoclonal antibody a2 were added into a reaction cell, an incubation reaction was performed for 30 min, and a AFP immune complex a4 labeled with copper ion materials at one end and biotin at the other end was formed;

(2) 10 μL of magnetic microspheres a5 with surface-labeled streptavidin were added into the mixture reacted from step (1), and the biotin labeled at one end of the AFP immune complex specifically bound to the streptavidin labeled on the surface of the magnetic microsphere, to form the magnetic microspheres a6 with surface-bound AFP immune complex;

(3) the nano-microspheres with surface-bound the AFP immune complex in step (2) were fixed to the surface of the working electrode (graphene electrode) a8 through a magnetic separation method, the residual liquid in the reaction cell a7 was removed, the nano-microspheres with surface-bound the AFP immune complex was washed 3 times with a PB buffer solution, and 50 μL of acetic acid electrolyte a9 was filled;

(4) a three-electrode system was used for the measurement: the working electrode (graphene electrode) a8, the counter electrode (platinum electrode) a10 and the reference electrode (calomel electrode) were correctly connected to the electrochemical workstation, and a voltammogram of copper ions on an immune complex on the surface of the magnetic microsphere was measured by utilizing a stripping voltammetry method; and then an actual characteristic peak within the range of +/−100 mV of the theoretical characteristic peak of the copper metal ions in the measured voltammogram was picked out, a half-peak area was calculated, a curve was fitted between the half-peak area and the concentration of the analyte by using a Log-Log regression equation to obtain a standard curve, and the content of the AFP was calculated by the standard curve.

6. Establishment of a Standard Curve:

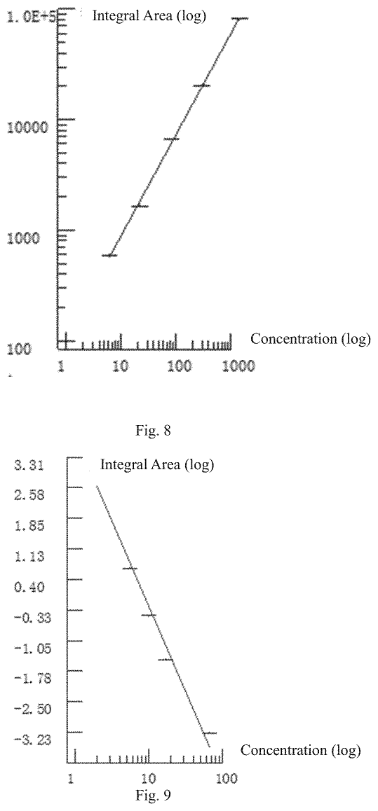

AFP calibrators at concentrations of 0, 5, 15, 50, 150, and 600 ng/mL were used to establish an AFP standard curve with a detection sensitivity of 5 ng/mL and a detection range of 5-600 ng/mL; the detection data is shown in Table 1, and the standard curve is shown in FIG. 8.

TABLE 1
Detection Data

| Standard concentration | |
|---|---|
| (ng/mL) | Integral area |
| 0 | 102 |
| 5 | 675 |
| 15 | 1825 |
| 50 | 7136 |
| 150 | 21270 |
| 600 | 81493 |

Method II: As Shown in FIGS. 3 and 4,

### Embodiment 2: Taking the Detection of Free Triiodothyronine (FT₃) as an Example

1. Preparation of Cadmium Ions-Embedded Polystyrene Microspheres:

10 g of styrene monomer, 1 g of sodium lauryl sulfate, 100 mL of purified water, and 1 g of cadmium chloride powder were stirred and mixed uniformly, placed in a 75° C. incubator, and sequentially mixed uniformly by introducing nitrogen gas; then 1 g of potassium persulfate was dissolved in 5 mL of purified water, the mixture was added to the reaction solution at a rate of 0.1 mL/min, and reacted under nitrogen for 50 min; after finishing the reaction, 100 mL of ice water mixture was added to rapidly reduce the temperature of the reaction solution to below 40° C. The solution was centrifuged 3 times at 10,000 r/min for 30 min each time, and dispersed and washed with 95% ethanol. Finally, the solid was dispersed with 100 mL of purified water; the dispersed material was placed in a 50° C. water bath for high-speed stirring, and a mixed acid of sulfuric acid and nitric acid (v/v=3:2) was added for reaction 2 h, and the reactant was centrifuged at 10,000 r/min to remove the supernatant, the precipitate was washed and dispersed with purified water to 100 mL, and this procedure was repeated 3 times. Finally, the dispersed material was transferred to a 75° C. incubator and 2 g of NaOH and 2 g of Na₂S₂O₄ were added, after reaction with stirring for 4 h, the solution was centrifuged at 10,000 r/min to remove the supernatant, the precipitate was washed and dispersed with purified water to 100 ml, the procedure was repeated 3 times, and the polystyrene microspheres containing Cd inside and on the surface were formed and recorded as cadmium ions materials.

2. A Cadmium Ion Materials-Labeled Mouse Anti-Human FT₃ Monoclonal Antibody:

Firstly, a mouse anti-human FT₃ monoclonal antibody was diluted to 1 mg/mL with sodium carbonate buffer 1 and dialyzed against sodium carbonate buffer 1 after stirring for 4 h at room temperature (25° C.±5° C.) in the dark; 1 mL of a mouse anti-human FT₃ monoclonal antibody solution was added to 5 mL of prepared cadmium ion materials, a magnetic stirring was performed at for 30 min 25° C.±5° C. in the dark, and then the mixed solution was transferred to a dialysis bag and dialyzed against a phosphate buffer at 4° C. overnight. Finally, The dialyzed substance was taken out and stored at −20° C. by adding the same amount of glycerol.

3. A Biotin-Labeled FT₃ Complete Antigen:

Firstly, a FT₃ complete antigen was diluted to 1 mg/mL with sodium carbonate buffer 1 and dialyzed against sodium carbonate buffer 1 after stirring for 4 h at room temperature (25° C.±5° C.) in the dark; 6-aminocaproic acid-N-hydroxysuccinimide-biotin (BCNHS) was prepared into 1 mg/mL solution with N,N-dimethyl amide (DMF); 80 μL of the above DMF solution was added to 1 mL of FT₃ complete antigen solution, the mixture was blended in a glass bottle, and stirred at room temperature (25° C.±5° C.) for 2 h in the dark; 9.6 μL of 1 mol/L ammonium chloride solution was added, and the mixture was stirred for 10 min at room temperature (25° C.±5° C.) in the dark; then the mixed solution was transferred to a dialysis bag and dialyzed against a phosphate buffer at 4° C. overnight. Finally, the dialyzed substance was taken out and stored at −20° C. by adding the same amount of glycerol.

4. Streptavidin-Labeled Magnetic Nano-Microspheres:

0.5 mL of 2.5 mg/mL magnetic microspheres (with a particle size of 800 nm, purchased from Tianjin Saierqun Technology Co., Ltd.) were washed twice with a phosphate buffer solution (pH=7.4) and then re-suspended in 10 mL of phosphate buffer solution, 4 mL of glutaraldehyde was sequentially added for shaking reaction for 10 h at room temperature, the reactant was washed with a large amount of purified water after finishing the reaction, and the magnetic microspheres were re-suspended in 10 mL of the phosphate buffer solution; 0.5 mg of streptavidin was dissolved in 1 mL of phosphate buffer solution, then the mixture was added into the phosphate buffer solution of the magnetic microspheres for shaking 8 h at room temperature, the reactant was washed with the phosphate buffer solution, and dispersed in 10 mL of phosphate buffer solution for later use.

5. Detection Process:

(1) 10 μL of a FT₃ b1-containing sample to be tested, 20 μL of cadmium ion materials-labeled mouse anti-human FT₃ monoclonal antibody b2 and 10 μL of another biotin-labeled FT₃ complete antigen b3 were added into a reaction cell b8, wherein the FT₃ in the sample competed with a biotin-labeled FT₃ complete antigen for an immunoreaction with a cadmium ions-labeled mouse anti-human FT₃ monoclonal antibody, and an incubation reaction was performed for 30 min, to form a first FT₃ immune complex b5 labeled with cadmium ions at one end and biotin at the other end and to form a second FT₃ immune complex b4 labeled with cadmium ions at one end;

(2) 10 μL of magnetic microspheres b6 with surface-labeled streptavidin were added into the mixture reacted from step (1), and the biotin labeled at one end of the first FT₃ immune complex specifically bound to the streptavidin labeled on the surface of the magnetic microsphere, to form the magnetic microsphere with surface-bound the first FT₃ immune complex b7;

(3) the magnetic microspheres with surface-bound the first FT₃ immune complex in step (2) were fixed to the surface of the working electrode (graphene electrode) b9 in a magnetic separation method, the residual liquid in the reaction cell b8 was removed, the magnetic microspheres with surface-bound the first FT₃ immune complex was washed 3 times with a PB buffer solution, and 50 μL of acetic acid electrolyte b10 was filled;

(4) a three-electrode system was used for the measurement; the working electrode (graphene electrode) b9, the counter electrode (platinum electrode) b11 and the reference electrode (calomel electrode) were correctly connected to the electrochemical workstation, and a voltammogram of cadmium ions on the first immune complex on the surface of the magnetic microspheres was measured by utilizing a stripping voltammetry method; and then an actual characteristic peak within the range of +/−100 mV of the theoretical characteristic peak of the cadmium ions in the measured voltammogram was picked out, a half-peak area was calculated, a curve was fitted between the half-peak area and the concentration of the FT₃ by using a Log-Logit regression equation to obtain a FT₃ standard curve, and the content of the FT₃ was calculated by the standard curve.

6. Establishment of Standard Curves

FT₃ calibrators at concentrations of 0, 1.8, 4.5, 7.5, 12, 40 pg/mL were used to establish a FT₃ standard curve. The voltammograms of Cd²⁺ measured after detection of different concentrations of FT₃ calibrators are shown in FIG. 7 with a detection sensitivity of 1.8 pg/mL, and a detection range of 1.8-40 pg/mL, the detection data are shown in Table 2, and the standard curve is shown in FIG. 9.

TABLE 2
Detection Data

| Standard concentration | |
|---|---|
| (pg/mL) | Integral area |
| 0 | 6549 |
| 1.8 | 6231 |
| 4.5 | 4326 |
| 7.5 | 2545 |
| 12 | 1197 |
| 40 | 245 |

Method II: As Shown in FIGS. 5 and 6,

### Embodiment 3: Taking the Detection of Free Triiodothyronine (FT₃) as an Example

1. Preparation of Cadmium Ions-Embedded Polystyrene Microspheres:

10 g of styrene monomer, 1 g of sodium lauryl sulfate, 100 mL of purified water, and 1 g of cadmium chloride powder were stirred and mixed uniformly, placed in a 75° C. incubator, and sequentially mixed uniformly by introducing nitrogen gas; then 1 g of potassium persulfate was dissolved in 5 mL of purified water, the mixture was added to the reaction solution at a rate of 0.1 mL/min, and reacted under nitrogen for 50 min; after finishing the reaction, 100 mL of ice water mixture was added to rapidly reduce the temperature of the reaction solution to below 40° C. The solution after reaction was centrifuged 3 times at 10,000 r/min for 30 min each time, and re-dispersed and washed with 95% ethanol. Finally, the solution was re-dispersed with 100 mL of purified water; re-dispersed material was placed in a 50° C. water bath for high-speed stirring, a mixed acid of sulfuric acid and nitric acid (v/v=3:2) was added and reacted for 2 h, the reactant was centrifuged at 10,000 r/min to remove the supernatant, the precipitate was washed and re-dispersed with purified water to 100 mL, and this procedure was repeated 3 times. Finally, the re-dispersed material was transferred to a 75° C. incubator and 2 g of NaOH and 2 g of Na₂S₂O₄ were added, after reaction with stirring for 4 h, the solution was centrifuged at 10,000 r/min to remove the supernatant, the precipitate was washed and re-dispersed with purified water to 100 mL, the procedure was repeated 3 times, and the polystyrene microsphere containing Cd²⁺ inside and on the surface was formed and recorded as cadmium ion materials.

2. A Cadmium Ion Materials-Labeled FT₃ Complete Antigen:

Firstly, a FT₃ complete antigen was diluted to 1 mg/mL with sodium carbonate buffer 1 and dialyzed against sodium carbonate buffer 1 after stirring for 4 h at room temperature (25° C.±5° C.) in the dark; 1 mL of FT₃ complete antigen solution was added to 5 mL of prepared cadmium ion materials, a magnetic stirring was performed at 25° C.±5° C. for 30 min in the dark, and then the mixed solution was transferred to a dialysis bag and dialyzed against a phosphate buffer at 4° C. overnight. Finally, the dialysate was taken out and stored at −20° C. by adding the same amount of glycerol.

3. A Biotin-Labeled Mouse Anti-Human FT₃ Monoclonal Antibody:

Firstly, a mouse anti-human FT₃ monoclonal antibody was diluted to 1 mg/mL with sodium carbonate buffer 1 and dialyzed against sodium carbonate buffer 1 after stirring for 4 h at room temperature (25° C.±5° C.) in the dark; 6-aminocaproic acid-N-hydroxysuccinimide-biotin (BCNHS) was prepared into 1 mg/mL solution with N,N-dimethyl amide (DMF); 80 μL of the above DMF solution was added to 1 mL of a mouse anti-human FT₃ monoclonal antibody solution, the mixture was blended in a glass bottle, and stirred for 2 h at room temperature (25° C.±5° C.) in the dark; 9.6 μL of 1 mol/L ammonium chloride solution was added, and the mixture was stirred for 10 min at room temperature (25° C.±5° C.) in the dark; then the mixed solution was transferred to a dialysis bag and dialyzed against a phosphate buffer at 4° C. overnight. Finally, the dialyzed substance was taken out and stored at −20° C. by adding the same amount of glycerol.

4. Streptavidin-Labeled Magnetic Nano-Microspheres:

0.5 mL of 2.5 mg/mL magnetic microspheres (with a particle size of 800 nm, purchased from Tianjin Saierqun Technology Co., Ltd.) were washed twice with a phosphate buffer solution (pH=7.4) and then re-suspended in 10 mL of phosphate buffer solution, 4 mL of glutaraldehyde was sequentially added for shaking reaction for 10 h at room temperature, the reactant was washed with a large amount of purified water after finishing the reaction, and the magnetic microsphere was re-suspended in 10 mL of phosphate buffer solution; 0.5 mg of streptavidin was dissolved in 1 mL of phosphate buffer solution, then the mixture was added into the phosphate buffer solution of the magnetic microspheres for shaking 8 h at room temperature, the reactant was washed with the phosphate buffer solution, and dispersed in 10 mL of phosphate buffer solution for later use.

5. Detection Process:

(1) 10 μL of a FT₃ c1-containing sample to be tested, 20 μL of a cadmium ion materials-labeled FT₃ complete antigen c2 and 10 μL of biotin-labeled mouse anti-human FT₃ monoclonal antibody c3 were added into a reaction cell c8, wherein the FT₃ in the sample competed with cadmium ion materials labeled FT₃ complete antigen for an immunoreaction with a biotin-labeled mouse anti-human FT₃ monoclonal antibody, and an incubation reaction was performed for 3-90 min, to form a first FT₃ immune complex c4 labeled with cadmium ion materials at one end and biotin at the other end and to form a second FT₃ immune complex c5 labeled with biotin at one end:

(2) 10 μL of magnetic microspheres c6 with surface-labeled streptavidin were added into the mixture reacted from step (1), and the biotins labeled at one end of the first and second FT₃ immune complex specifically bound to the streptavidins labeled on the surface of the magnetic microspheres, to form the magnetic microsphere c7 with surface-bound the first and second FT₃ immune complex;

(3) the magnetic-microspheres with surface-bound the first and second FT₃ immune complex in step (2) were fixed to the surface of the working electrode (graphene electrode) c9 in a magnetic separation method, the residual liquid in the reaction cell c8 was removed, the electrode was washed 3 times with a PB buffer solution, and 50 μL of acetic acid electrolyte c10 was filled:

(4) a three-electrode system was used for the measurement: the working electrode (graphene electrode) c9, the counter electrode (platinum electrode) c11 and the reference electrode (calomel electrode) were correctly connected to the electrochemical workstation, and a voltammogram of cadmium ions on the first FT₃ immune complex on the surface of magnetic microspheres was measured by utilizing a stripping voltammetry method; and then an actual characteristic peak within the range of +/−100 mV of the theoretical characteristic peak of the cadmium ions in the measured voltammogram was picked out, a half-peak area was calculated, a curve was fitted between the half-peak area and the concentration of the FT₃ by using a Logit-Log regression equation to obtain a FT₃ standard curve, and the content of the FT₃ was calculated by the standard curve.

6. Establishment of a Standard Curve

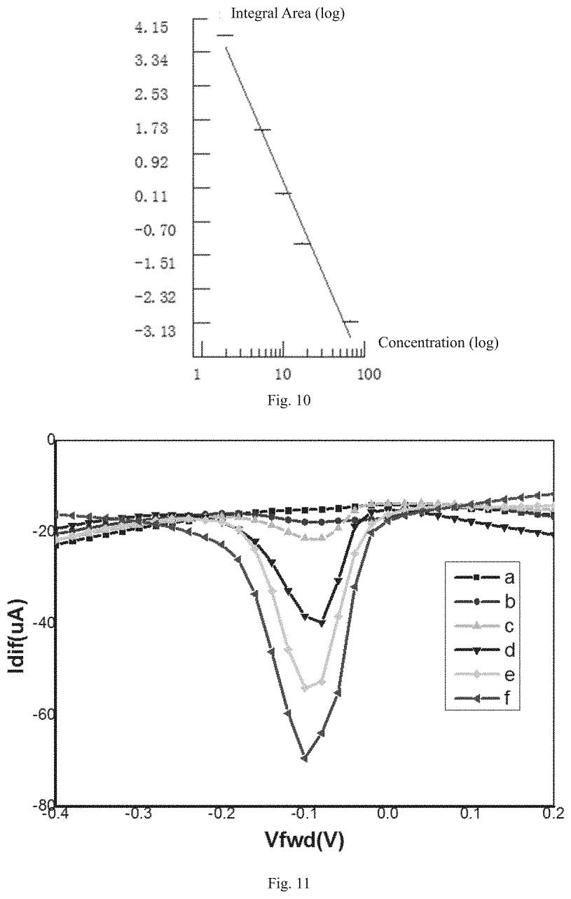

FT₃ calibrators at concentrations of 0, 1.8, 4.5, 7.5, 12, 40 pg/mL were used to establish a FT₃ standard curve with a detection sensitivity of 1.8 pg/mL, and a detection range of 1.8-40 pg/mL, the detection data are shown in Table 3, and the standard curve is shown in FIG. 10.

TABLE 3
Detection Data

| Standard concentration | |
|---|---|
| (pg/mL) | Integral area |
| 0 | 12933 |
| 1.8 | 12642 |
| 4.5 | 10539 |
| 7.5 | 6362 |
| 12 | 2937 |
| 40 | 563 |

### Embodiment 4: Taking the Detection of Procalcitonin (PCT) as an Example (a Sandwich Method)

1. A Nano Copper Oxide-Labeled Mouse Anti-Human PCT Monoclonal Antibody:

A mouse anti-human PCT monoclonal antibody was diluted into 1 mg/mL with a PBS buffer solution, and dialyzed against PBS buffer solution after stirring for 4 h at room temperature (25° C.±5° C.) in the dark for later use; 1 mL of mouse anti-human PCT monoclonal antibody solution was added to 1-10 mL (preferably 5 mL) of prepared nano copper oxide (1-100 nm, preferably 30-40 nm) material (mass fraction 1 g/100 mL, purity 99.9/6), the mixture was magnetically stirred for 30 min at 25° C.±5° C. in the dark; the supernatant was then removed by centrifugation at 10,000 r/min for 5-30 min (preferably 10 min), and the precipitate was re-dispersed and blocked with 10 mL of 1-10 wt % (preferably 10 wt %) bovine serum albumin (BSA) for 1-5 h (preferably 3 h), followed by centrifugation at 3000-8000 r/min (preferably 5000 r/min) for 5-30 min (preferably 10 min), and finally the supernatant was removed and the precipitate was taken out for storage at 2-8° C. The working solution was diluted 100-5000 times (preferably 300 times) with a PBS buffer for later use. The labeling effect was better and more stable with the preferred solution.

2. Another Biotin-Labeled Mouse Anti-Human PCT Monoclonal Antibody:

Another mouse anti-human PCT monoclonal antibody was diluted into 1 mg/mL with sodium carbonate buffer solution 2, and dialyzed against sodium carbonate buffer solution 2 after stirring for 1-5 h (preferably 4 h) at room temperature (25° C.±5° C.) in the dark; then 6-aminocaproic acid-N-hydroxysuccinimide-biotin (BCNHS) was prepared into 1 mg/mL solution by using N,N-dimethylamide (DMF); 10-160 μL (preferably 80 μL) of above DMF solution was added to 1 mL of another mouse anti-human PCT monoclonal antibody solution, the mixture was blended in a glass bottle, and stirred for 1-5 h (preferably 2 h) at room temperature (25° C.±5° C.) in the dark; 5-20 μL (preferably 9.6 μL) of 1 mol/L ammonium chloride solution was added to the above mixture, and stirred for 10 min at room temperature (25° C.±5° C.) in the dark; the mixed solution was then transferred to a dialysis bag and dialyzed overnight against a phosphate buffer at 4° C. Finally, the dialyzed substance was taken out and stored at −20° C. by adding the same amount of glycerol. The working solution was diluted 100-5000 times (preferably 300 times) with a PBS buffer for later use. The labeling effect was better and more stable with the preferred solution.

3. Streptavidin-Labeled Magnetic Microspheres:

0.5 mL of 2.5 mg/mL magnetic microspheres (with a particle size of 300 nm-3000 nm, preferably 800 nm, purchased from Sigma) were washed twice with a phosphate buffer solution (pH=7.4) and then re-suspended in 10 mL of phosphate buffer solution, 4 mL of glutaraldehyde was sequentially added for shaking reaction for 10 h at room temperature, the reactant was washed for 2-3 times with purified water after finishing the reaction, and the magnetic microsphere was re-suspended in 10 mL of the phosphate buffer solution; 0.5 mg of streptavidin was dissolved in 1 mL of phosphate buffer solution, the mixture was added into the phosphate buffer solution of the magnetic microsphere and shaked for 8 h at room temperature, the reactant was washed 2-3 times with the phosphate buffer solution after finishing the reaction, and then dispersed in 10 mL of phosphate buffer solution for later use. The labeling effect was better and more stable with the preferred solution.

4. The Detection Process was as Follows:

(1) 10-100 μL (preferably 25 μL) of PCT-containing sample to be tested, 10-150 μL (preferably 100 μL) of nano copper oxide-labeled mouse anti-human PCT monoclonal antibody and 10-150 μL (preferably 50 μL) of another biotin-labeled mouse anti-human PCT monoclonal antibody were added into a reaction cell and incubated for 3-90 min (preferably 15 min) to form a PCT immune complex labeled with nano copper oxide at one end and with biotin at the other end;

(2) 10-100 μL (preferably 50 μL) magnetic microspheres with surface-labeled streptavidin were added into the mixture reacted from step (1), and the biotin labeled at one end of the PCT immune complex specifically bound to the streptavidin labeled on the surface of the magnetic microspheres, to form the magnetic microspheres with surface-bound the PCT immune complex;

(3) the magnetic-microspheres with surface-bound the PCT immune complex in step (2) were fixed to the surface of the working electrode in a magnetic separation method, the residual liquid in the reaction cell was removed, and 25-1000 μL (preferably 100 μL) of citric acid electrolyte was filled;

(4) a four-electrode system was used for the measurement; the electrochemical detection was performed on screen printing electrodes of a working electrode (graphite electrode), an internal control electrode (carbon electrode), a counter electrode (platinum electrode or carbon electrode) and a reference electrode (Ag/AgCl electrode). Firstly, the internal control electrode, the counter electrode and the reference electrode were connected to the electrochemical workstation, the voltammetric curve of the substrate was determined for cardinality calibration, the working electrode, counter electrode, and reference electrode were connected to the electrochemical workstation, and a voltammogram of reduction reaction of nano copper oxide on the immune complex on the surface of the magnetic microspheres was measured by utilizing a voltammetry method; and then the theoretical characteristic peak of copper metal ion in the measured voltammogram was picked out, a half-peak area was calculated, a curve was fitted between the half-peak area and the concentration of the PCT by using a Log-Log linear regression equation or a four-parameter equation (preferably a four-parameter equation) to obtain a PCT standard curve, and the content of the PCT was calculated by the standard curve.

And the detection sensitivity was higher and the stability was better by adopting the preferred solution in each step.

5. Establishment of a Standard Curve:

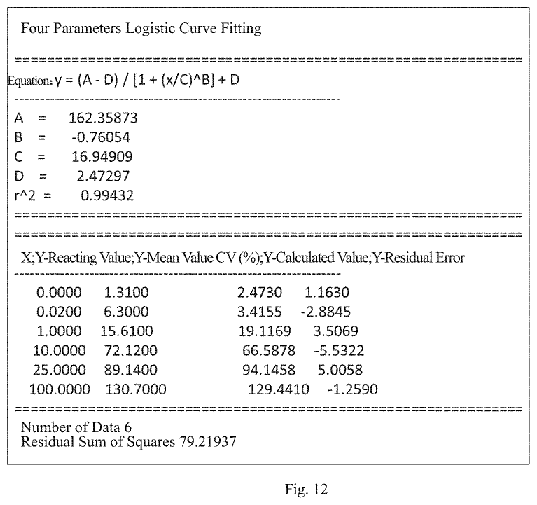

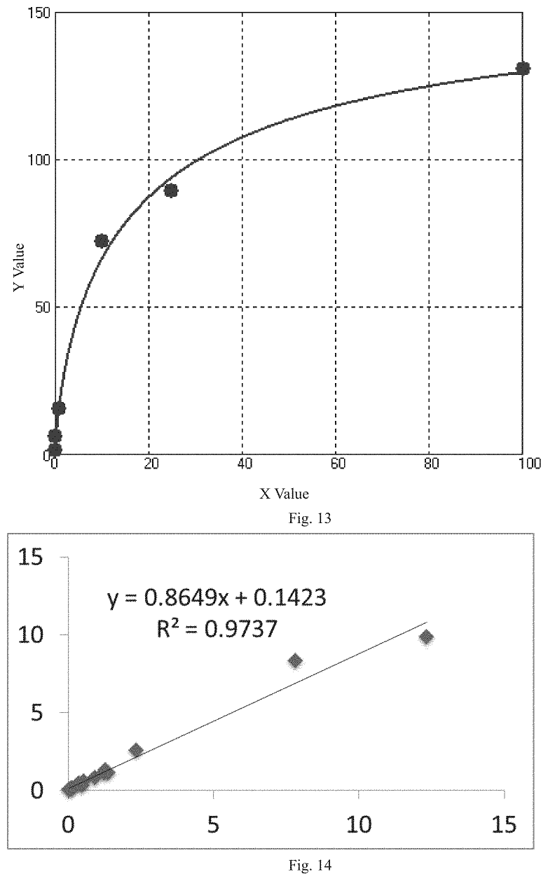

PCT calibrators at concentrations of 0, 0.02, 1, 10, 25, 100 ng/mL were used to establish a PCT standard curve, with a detection sensitivity of 0.02 ng/mL, and a detection range of 0.02-100 ng/mL, the voltammogram of copper ions is shown in FIG. 11, the detection data are shown in table 4, the four-parameter logistic curve fitting data are shown in FIG. 12, and the standard curve is shown in FIG. 13.

TABLE 4
Detection Data

| Standard concentration | |
|---|---|
| (ng/mL) | Integral area |
| 0 | 1.31 |
| 0.02 | 6.30 |
| 1 | 15.61 |
| 10 | 72.12 |
| 25 | 89.14 |
| 100 | 130.70 |

6. Comparison with Roche's Results:

13 samples were detected by the method of the present invention and Roche's electrochemiluminescence method; the detection results are shown in Table 5. The correlation analysis of the detection results is shown in FIG. 14, and the regression equation is y=0.86498x+0.1423, R²=0.9737, indicating that the method has a good correlation with Roche's electrochemiluminescence method.

TABLE 5
Comparison of the results of this method with
that of the Roche's electrochemical method

| This method (ng/mL) | Roche's (ng/mL) |
|---|---|
| 0.11 | 0.15 |
| 12.33 | 9.87 |
| 1.23 | 1.13 |
| 0.53 | 0.62 |
| 7.81 | 8.32 |
| 0.02 | 0.04 |
| 1.28 | 1.31 |
| 0.49 | 0.32 |
| 0.16 | 0.15 |
| 0.91 | 0.82 |
| 2.34 | 2.59 |
| 1.37 | 1.12 |
| 0.37 | 0.45 |

### Embodiment 5: Taking the Detection of Free Thyroxine (FT₄) as an Example (a Competition Method)

1. A Nano Copper Oxide-Labeled FT₄ Complete Antigen:

An FT₄ complete antigen was diluted into 1 mg/mL with a PBS buffer solution, and dialyzed against PBS buffer solution after stirring for 4 h at room temperature (25° C.±5° C.) in the dark for later use; 1 mL of FT₄ complete antigen solution was added to 1-10 mL (preferably 2 mL) of prepared nano copper oxide (1-100 nm, preferably 30-40 nm) material (mass fraction 1 g/100 mL, purity 99.9%), the mixture was magnetically stirred for 30 min at 25° C. ° C. in the dark; the supernatant was then removed by centrifugation at 10,000 r/min for 5-30 min (preferably 10 min), and the precipitate was re-dispersed and blocked with 10 mL of 1-10% (preferably 10%) BSA for 1-5 h (preferably 3 h), followed by centrifugation at 3000-8000 r/min (preferably 5000 r/min) for 5-30 min (preferably 10 min), and finally the supernatant was removed and the precipitate was taken out for storage at 2-8° C. The working solution was diluted 100-5000 times (preferably 300 times) with a PBS buffer for later use. The labeling effect was better and more stable with the preferred solution.

2. Another Biotin-Labeled Goat Anti-Human FT₄ Polyclonal Antibody:

Another goat anti-human FT₄ polyclonal antibody was diluted into 1 mg/mL with sodium carbonate buffer solution 2, and dialyzed against sodium carbonate buffer solution 2 after stirring for 1-5 h (preferably 4 h) at room temperature (25° C.±5° C.) in the dark; then 6-aminocaproic acid-N-hydroxysuccinimide-biotin (BCNHS) was prepared into 1 mg/mL solution by using N,N-dimethylamide (DMF); 10-160 μL (preferably 40 μL) of above DMF solution was added to 1 mL of goat anti-human FT₄ polyclonal antibody solution, the mixture was blended in a glass bottle, and stirred for 1-5 h (preferably 2 h) at room temperature (25° C.±5° C.) in the dark; 5-20 μL (preferably 15 μL) of 1 mol/L ammonium chloride solution was added to the above mixture, and stirred for 10 min at room temperature (25° C.±5° C.) in the dark; the mixed solution was then transferred to a dialysis bag and dialyzed against a phosphate buffer at 4° C. overnight. Finally, the dialyzed substance was taken out and stored at −20° C. by adding the same amount of glycerol. The working solution was diluted 100-5000 times (preferably 300 times) with a PBS buffer for later use. The labeling effect was better and more stable with the preferred solution.

3. Streptavidin-Labeled Magnetic Microspheres:

0.5 mL of 2.5 mg/mL magnetic microspheres (with a particle size of 300 nm-3000 nm, preferably 800 nm, purchased from Sigma) were washed twice with a phosphate buffer solution (pH=7.4) and then re-suspended in 10 mL of the phosphate buffer solution, 4 mL of glutaraldehyde was sequentially added for shaking reaction for 10 h at room temperature, the reactant was washed for 2-3 times with purified water after finishing the reaction, and the magnetic microspheres were re-suspended in 10 mL of phosphate buffer solution; 0.5 mg of streptavidin was dissolved in 1 mL of phosphate buffer solution, the mixture was added into the phosphate buffer solution of the magnetic microsphere for shaking 8 h at room temperature, the reactant was washed 2-3 times with the phosphate buffer solution after finishing the reaction, and then dispersed in 10 mL of phosphate buffer solution for later use. The labeling effect was better and more stable with the preferred solution.

4. The Detection Process was as Follows:

(1) 10-100 μL (preferably 50 μL) of FT₄-containing sample to be tested, 10-150 μL (preferably 50 μL) of nano copper oxide-labeled FT₄ complete antigen and 10-150 μL (preferably 50 μL) of biotin-labeled goat anti-human FT₄ polyclonal antibody were added into a reaction cell and incubated for 3-90 min (preferably 15 min), wherein the FT₄ in the sample to be tested competed with the nano copper oxide-labeled FT₄ complete antigen for an immunoreaction with a biotin-labeled goat anti-human FT₄ polyclonal antibody to form an FT₄ immune complex I labeled with copper oxide at one end and with biotin at the other end, to form an FT₄ immune complex 11 labeled with biotin at one end:

(2) 10-100 μL (preferably 50 μL) magnetic microspheres with surface-labeled streptavidin were added into the mixture reacted from step (1), and the biotins labeled at one end of the FT₄ immune complex 1 and FT₄ immune complex II specifically bound to the streptavidins labeled on the surface of the magnetic microspheres, to form the magnetic microspheres with surface-bound the FT₄ immune complex I and FT₄ immune complex H;

(3) the magnetic microspheres with surface-bound the FT₄ immune complex I and FT₄ immune complex 11 in step (2) were fixed to the surface of the working electrode in a magnetic separation method, the residual liquid in the reaction cell was removed, and 25-1000 μL (preferably 100 μL) of citric acid electrolyte was filled;

(4) a four-electrode system was used for the measurement; the electrochemical detection was performed on screen printing electrodes of a working electrode (gold microelectrode), an internal control electrode (silver electrode), a counter electrode (platinum electrode or carbon electrode) and a reference electrode (Ag/AgCl electrode). Firstly, the internal control electrode, the counter electrode and the reference electrode were connected to the electrochemical workstation, a voltammogram of the substrate was measured, the cardinality calibration was performed, and then the working electrode, the counter electrode and the reference electrode were connected to the electrochemical workstation, and a voltammogram of reduction reaction of nano copper oxide on the immune complex on the surface of the magnetic microspheres was measured by utilizing a voltammetry method, then characteristic peak of copper metal ions in the measured voltammogram was picked out, a half-peak area was calculated, an curve was fitted between the half-peak area and the concentration of the FT₄ by using a Log-Logit linear regression equation or a four-parameter equation (preferably a four-parameter equation) to obtain an FT₄ standard curve, and the content of the FT₄ was calculated by the standard curve.

And the detection sensitivity was higher and the stability was better by adopting the preferred solution in each step.

5. Establishment of a Standard Curve:

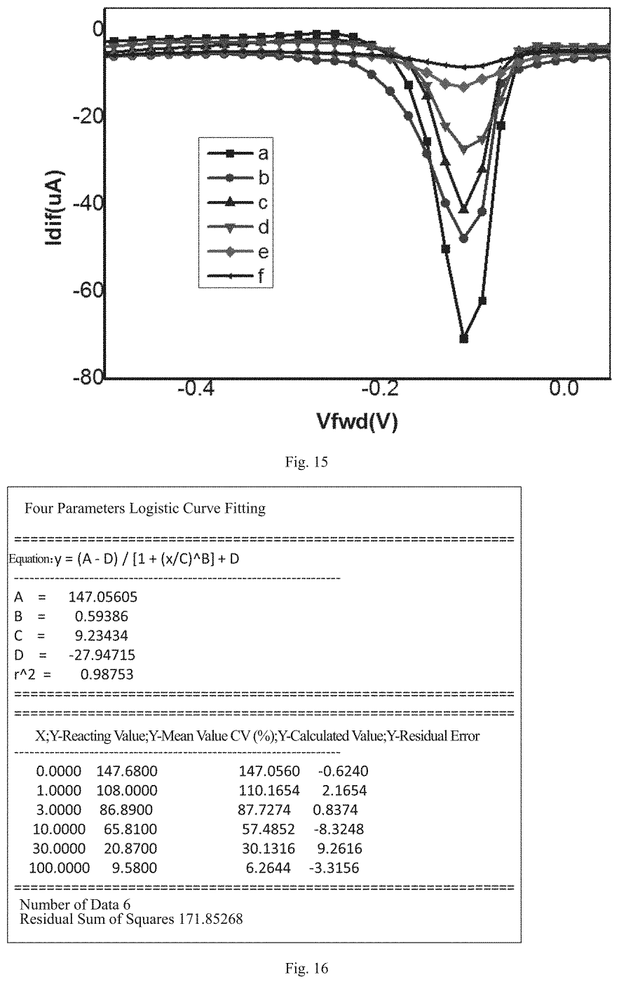

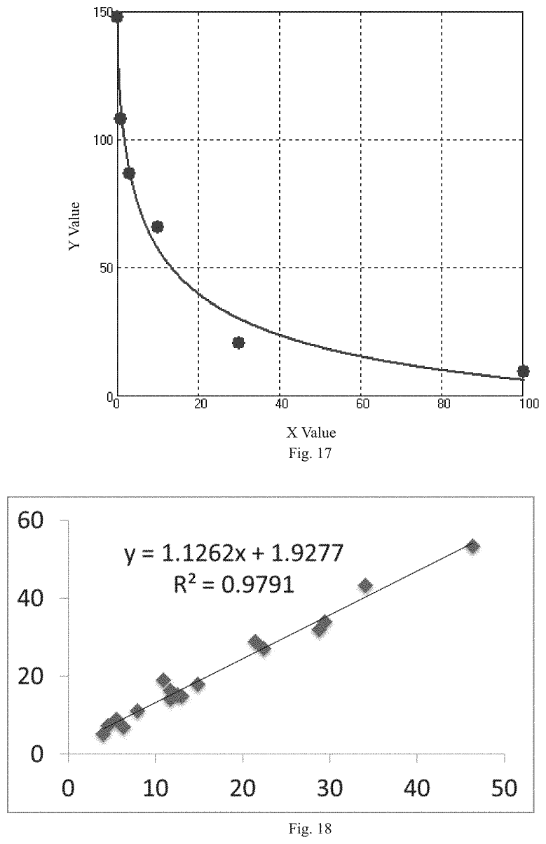

FT₄ calibrators at concentrations of 0, 1, 3, 10, 30, 100 pg/mL were prepared to establish an FT₄ standard curve, with a detection sensitivity of 0.5 pg/mL, and a detection range of 1-100 pg/mL, the voltammogram of copper ions is shown in FIG. 15, the detection data are shown in Table 6, the four-parameter logistic curve fitting data are shown in FIG. 16, and the standard curve is shown in FIG. 17.

TABLE 6
Detection Data

| Standard concentration | |
|---|---|
| (pg/mL) | Integral area |
| 0 | 147.68 |
| 1 | 108.00 |
| 3 | 86.89 |
| 10 | 65.81 |
| 30 | 20.87 |
| 100 | 9.58 |

6. Comparison with Roche's Results:

17 samples were detected by the method of the present invention and Roche's electrochemiluminescence method; the detection results are shown in Table 7. The correlation analysis of the detection results is shown in FIG. 18, and the regression equation is y=1.1262x+1.9277, R²=0.9797, indicating that the method has a good correlation with Roche's electrochemiluminescence method.

TABLE 7
Comparison of the results of this method with
that of the Roche's electrochemical method

| This method (pg/mL) | Roche's (pmol/L) |
|---|---|
| 4.02 | 5.14 |
| 13.06 | 14.89 |
| 10.95 | 18.98 |
| 46.40 | 53.29 |
| 6.33 | 6.97 |
| 5.55 | 8.92 |
| 11.72 | 14.02 |
| 14.86 | 17.83 |
| 11.72 | 16.27 |
| 29.48 | 33.87 |
| 22.48 | 27.03 |
| 7.94 | 11.12 |
| 12.56 | 15.21 |
| 34.09 | 43.29 |
| 21.48 | 28.93 |
| 28.79 | 31.82 |
| 4.60 | 7.32 |

### Embodiment 6: Taking the Detection of Ferritin (Fer) as an Example (Sandwich Method)

1. A Nano Copper Oxide-Labeled Mouse Anti-Human Fer Monoclonal Antibody:

A mouse anti-human Fer monoclonal antibody was diluted into 1 mg/mL with a PBS buffer solution, and dialyzed against the PBS buffer solution after stirring for 4 h at room temperature (25° C.±5° C.) in the dark for later use; 1 mL of a mouse anti-human Fer monoclonal antibody solution was added to 1-10 mL (preferably 5 mL) of prepared nano copper oxide (1-100 nm, preferably 30-40 nm) material (mass fraction 1 g/100 mL, purity 99.9%), the mixture was magnetically stirred for 30 min at 25° C.±5° C. in the dark; the supernatant was then removed by centrifugation at 10,000 r/min for 5-30 min (preferably 10 min), and the precipitate was re-dispersed and blocked with 10 mL of 1-10% (preferably 10%) BSA for 1-5 h (preferably 3 h), followed by centrifugation at 3000-8000 r/min (preferably 6000 r/min) for 5-30 min (preferably 15 min), and finally the supernatant was removed and the precipitate was taken out for storage at 2-8° C. The working solution was diluted 100-5000 times (preferably 2000 times) with a PBS buffer for later use. The labeling effect was better and more stable with the preferred solution.

2. Another Magnetic Microspheres-Labeled Mouse Anti-Human Fer Monoclonal Antibody:

0.5 mL of 2.5 mg/mL magnetic microspheres (with a particle size of 300 nm-3000 nm, preferably 800 nm, purchased from Sigma) were washed twice with a phosphate buffer solution (pH=7.4) and then re-suspended in 10 mL of the phosphate buffer solution, 4 mL of glutaraldehyde was sequentially added for shaking reaction for 10 h at room temperature, the reactant was washed for 2-3 times with purified water after finishing the reaction, and the magnetic microspheres were re-suspended in 10 mL of the phosphate buffer solution; another mouse anti-human Fer monoclonal antibody was diluted into 1 mg/mL with sodium carbonate buffer solution 2, and dialyzed against sodium carbonate buffer solution 2 after stirring for 1-5 h (preferably 2 h) at room temperature (25° C.±5° C.) in the dark; 0.5-5 mL (preferably 1 mL) of another mouse anti-human Fer monoclonal antibody was pipetted and added into 10 mL of phosphate buffer solution of the magnetic microspheres for shaking for 8 h at room temperature; the magnetic microspheres were sucked on one side by using a magnet, washed 3 times by using PBS, and then dispersed in 10 mL of phosphate buffer solution. The labeling effect was better and more stable with the preferred solution.

3. The Detection Process was as Follows:

(1) 10-100 μL (preferably 25 μL) of Fer-containing sample to be tested, 10-150 μL (preferably 100 μL) of a nano copper oxide-labeled mouse anti-human Fer monoclonal antibody and 10-150 μL (preferably 50 μL) of another magnetic microspheres-labeled mouse anti-human Fer monoclonal antibody were added into a reaction cell and incubated for 3-90 min (preferably 10 min) to form a Fer immune complex labeled with nano copper oxide at one end and with magnetic microsphere at the other end;

(2) the magnetic microspheres with surface-bound Fer immune complex in step (1) were fixed to the surface of a working electrode coated with graphite or fullerene by adopting a magnetic separation method, then residual liquid in the reaction cell was removed, and 25-1000 μL (preferably 100 uL) citric acid electrolyte was filled;

(3) a four-electrode system was used for the measurement; the electrochemical detection was performed on screen printing electrodes of a working electrode (carbon electrode), an internal control electrode (graphite electrode), a counter electrode (platinum electrode or carbon electrode) and a reference electrode (Ag/AgCl electrode). Firstly, the internal control electrode, the counter electrode and the reference electrode were connected to the electrochemical workstation, a voltammogram of the substrate was measured, the cardinality calibration was performed, and then the working electrode, the counter electrode and the reference electrode were connected to the electrochemical workstation, and a voltammogram of reduction reaction of nano copper oxide on the immune complex on the surface of the magnetic microspheres was measured by utilizing a voltammetry method; and then characteristic peak of copper ions in the measured voltammogram was picked out, a half-peak area was calculated, an curve was fitted between the half-peak area and the concentration of the Fer by using a Log-Log linear regression equation or a four-parameter equation (preferably a four-parameter equation) to obtain an Fer standard curve, and the content of the Fer was calculated by the standard curve.

And the detection sensitivity was higher and the stability was better by adopting the preferred solution in each step.

4. Establishment of a Standard Curve:

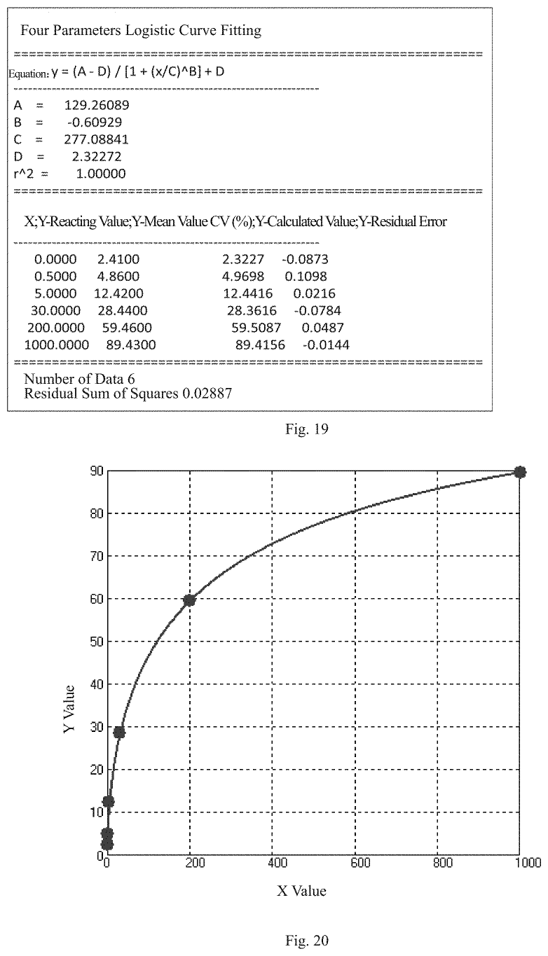

Fer calibrators at concentrations of 0, 0.5, 5, 30, 200, 1000 ng/mL were used to establish a Fer standard curve with a detection sensitivity of 0.1 ng/mL and a detection range of 0.5-1000 ng/mL. The detection data are shown in Table 8, and the four-parameter logistic curve fitting data are shown in FIG. 19 and the standard curve is shown in FIG. 20.

TABLE 8
Detection Data

| Standard concentration | |
|---|---|
| (ng/mL) | Integral area |
| 0 | 2.41 |
| 0.5 | 4.86 |
| 5 | 12.42 |
| 30 | 28.44 |
| 200 | 59.46 |
| 1000 | 89.43 |

5. Comparison with Roche's Results:

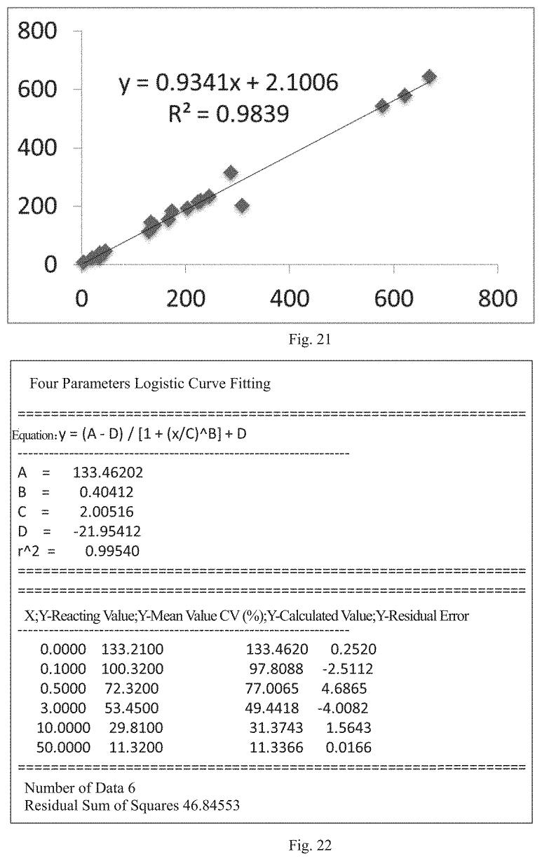

20 samples were detected by adopting the method of the present invention and Roche's electrochemiluminescence method; the detection results are shown in Table 9, the correlation analysis of the detection results is shown in FIG. 21, the regression equation is y=0.9341x+2.1006, and R²=0.9839, indicating that the method has good correlation with the Roche's electrochemiluminescence method.

TABLE 9
Comparison of the results of this method with
that of the Roche's electrochemical method

| This method (ng/mL) | Roche's (ng/mL) |
|---|---|
| 33.87 | 26.74 |
| 287.43 | 314.34 |
| 622.03 | 578.03 |
| 140.24 | 134.23 |
| 166.75 | 153.87 |
| 20.34 | 23.45 |
| 45.92 | 45.94 |
| 4.43 | 7.32 |
| 245.93 | 234.33 |
| 129.03 | 114.23 |
| 134.24 | 145.23 |
| 229.32 | 218.93 |
| 669.02 | 643.08 |
| 578.45 | 543.85 |
| 308.75 | 203.13 |
| 34.98 | 39.32 |
| 34.88 | 29.43 |
| 173.92 | 183.54 |
| 203.45 | 192.34 |
| 224.08 | 215.32 |

### Embodiment 7: Taking the Detection of Free Triiodothyronine (FT₃) as an Example (a Competition Method)

1. A Nano Copper Oxide-Labeled FT₃ Complete Antigen:

An FT₃ complete antigen was diluted into 1 mg/mL with a PBS buffer solution, and dialyzed against PBS buffer solution after stirring for 4 h at room temperature (25° C.±5° C.) in the dark for later use; 1 mL of FT₃ complete antigen solution was added to 1-10 mL (preferably 2 mL) of prepared nano copper oxide (1-100 nm, preferably 30-40 nm) material (mass fraction 1 g/100 mL, purity 99.9%), the mixture was magnetically stirred for 30 min at 25° C.±5° C. in the dark; the supernatant was then removed by centrifugation at 10,000 r/min for 5-30 min (preferably 10 min), and the precipitate was re-dispersed and blocked with 10 mL of 1-10% (preferably 10%) BSA for 1-5 h (preferably 3 h), followed by centrifugation at 3000-8000 r/min (preferably 6000 r/min) for 5-30 min (preferably 15 min), and finally the supernatant was removed and the precipitate was taken out for storage at 2-8° C. The working solution was diluted 100-5000 times (preferably 200 times) with a PBS buffer for later use. The labeling effect was better and more stable with the preferred solution.

2. Another Magnetic Microspheres-Labeled Goat Anti-Human FT₃ Polyclonal Antibody:

0.5 mL of 2.5 mg/mL magnetic microspheres (with a particle size of 300 nm-3000 nm, preferably 800 nm, purchased from Sigma) were washed twice with a phosphate buffer solution (pH=7.4) and then re-suspended in 10 mL of the phosphate buffer solution, 4 mL of glutaraldehyde was sequentially added for shaking reaction for 10 h at room temperature, the reactant was washed for 2-3 times with purified water after finishing the reaction, and the magnetic microsphere was re-suspended in 10 mL of the phosphate buffer solution; another goat anti-human FT₃ polyclonal antibody was diluted into 1 mg/mL with sodium carbonate buffer solution 2, and dialyzed against sodium carbonate buffer solution 2 after stirring for 1-5 h (preferably 2 h) at room temperature (25° C.±5° C.) in the dark; 0.5-5 mL (preferably 1 mL) of another goat anti-human FT₃ polyclonal antibody was pipetted and added into 10 mL of phosphate buffer solution of the magnetic microspheres for shaking for 8 h at room temperature; the magnetic microspheres were sucked on one side by using a magnet, washed 3 times by using PBS, and then dispersed in 10 mL of phosphate buffer solution. The labeling effect was better and more stable with the preferred solution.

3. The Detection Process was as Follows:

(1) 10-100 μL (preferably 50 μL) of FT₃-containing sample to be tested, 10-150 μL (preferably 100 μL) of nano copper oxide-labeled FT₃ complete antigen and 10-150 μL (preferably 50 μL) of another magnetic microspheres-labeled goat anti-human FT₃ polyclonal antibody were added into a reaction cell and incubated for 3-90 min (preferably 15 min), wherein the FT₃ in the sample to be tested competed with the nano copper oxide-labeled FT₃ complete antigen for an immunoreaction with another magnetic microsphere-labeled goat anti-human FT₃ polyclonal antibody, to form the magnetic microspheres with surface-bound the immune complex I with FT₃ in the sample to be tested and immune complex II with a nano copper oxide-labeled FT₃ complete antigen.

(2) the magnetic microspheres with surface-bound the immune complex I and immune complex 11 in step (1) were fixed to the surface of the working electrode in a magnetic separation method, the residual liquid in the reaction cell was removed, and 25-1000 μL (preferably 100 μL) of citric acid electrolyte was filled;

(3) a four-electrode system was used for the measurement; the electrochemical detection was performed on screen printing electrodes of a working electrode (glassy carbon microelectrode), an internal control electrode (carbon electrode), a counter electrode (platinum electrode or carbon electrode) and a reference electrode (Ag/AgCl electrode). Firstly, the internal control electrode, the counter electrode and the reference electrode were connected to the electrochemical workstation, a voltammogram of the substrate was measured, the cardinality calibration was performed, and then the working electrode, the counter electrode and the reference electrode were connected to the electrochemical workstation, and a voltammogram of reduction reaction of nano copper oxide on the immune complex on the surface of the magnetic microspheres was measured by utilizing a voltammetry method; and then characteristic peak of copper ions in the measured voltammogram was picked out, a half-peak area was calculated, an curve was fitted between the half-peak area and the concentration of the FT₃ by using a Log-Logit linear regression equation or a four-parameter equation (preferably a four-parameter equation) to obtain FT₃ standard curve, and the content of the FT₃ was calculated by the standard curve.

And the detection sensitivity was higher and the stability was better by adopting the preferred solution in each step.

4. Establishment of a Standard Curve:

FT₃ calibrators at concentrations of 0, 0.1, 0.5, 3, 10, 50 pg/mL were used to establish an FT₃ standard curve with a detection sensitivity of 0.05 pg/mL and a detection range of 0.1-50 pg/mL. The detection data are shown in Table 10. A four-parameter Logistic curve fitting data is shown in FIG. 22 and a standard curve is shown in FIG. 23.

TABLE 10
Detection Data

| Standard concentration | |
|---|---|
| (pg/mL) | Integral area |
| 0 | 132.31 |
| 0.1 | 100.32 |
| 0.5 | 72.32 |
| 3 | 53.45 |
| 10 | 29.81 |
| 50 | 11.32 |

6. Comparison with Roche's Results:

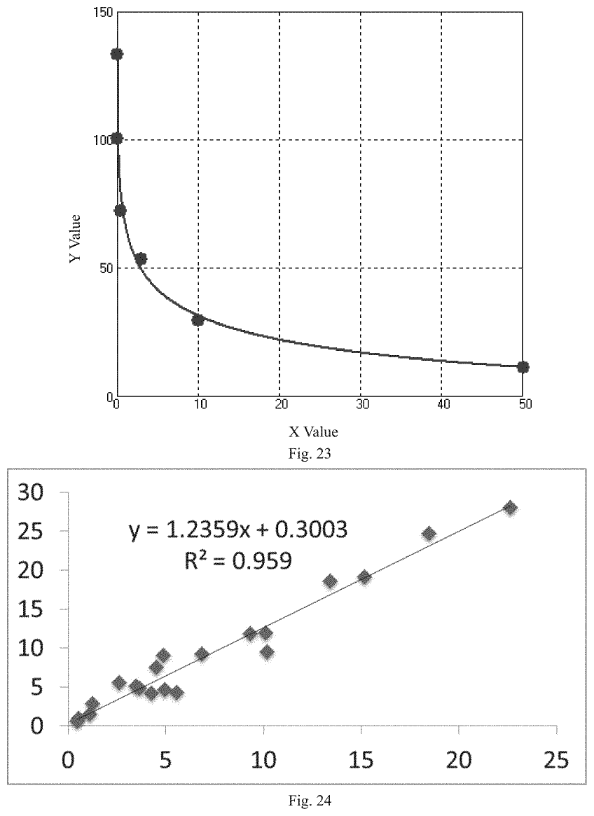

20 samples were detected by adopting the method of the present invention and Roche's electrochemiluminescence method; the detection results are shown in Table 11, the correlation analysis of the detection results is shown in FIG. 24, the regression equation is y=1.2359x+0.3003, and R²=0.959, indicating that the method has good correlation with the Roche's electrochemiluminescence method.

TABLE 11
Comparison of the results of this method with
that of the Roche's electrochemical method

| New method (pg/mL) | Roche's (pmol/L) |
|---|---|
| 22.65 | 28.02 |
| 3.66 | 4.8 |
| 3.50 | 5.07 |
| 13.44 | 18.57 |
| 4.29 | 4.15 |
| 1.26 | 2.81 |
| 4.53 | 7.53 |
| 10.13 | 11.92 |
| 10.20 | 9.46 |
| 1.11 | 1.46 |
| 0.47 | 0.54 |
| 6.86 | 9.24 |
| 5.58 | 4.22 |
| 2.61 | 5.56 |
| 0.53 | 0.97 |
| 4.96 | 4.63 |
| 4.90 | 9.05 |
| 9.33 | 11.81 |
| 15.18 | 19.12 |
| 18.49 | 24.67 |

### Embodiment 8: Taking the Detection of Human Epididymal Protein 4 (HE4) as an Example (a Sandwich Method)

1. Nano Copper Oxide-Labeled Streptavidin:

Streptavidin was diluted into 1 mg/mL with a PBS buffer solution, and dialyzed against PBS buffer solution after stirring for 4 h at room temperature (25° C.±5° C.) in the dark for later use; 1 mL of streptavidin solution was added to 1-10 mL (preferably 3 mL) of prepared nano copper oxide (1-100 nm, preferably 30-40 nm) material (mass fraction 1 g/100 mL, purity 99.9%), the mixture was magnetically stirred for 30 min at 25° C.±5° C. in the dark; the supernatant was then removed by centrifugation at 10,000 r/min for 5-30 min (preferably 15 min), and the precipitate was re-dispersed and blocked with 10 mL of 1-10% (preferably 10%) BSA for 1-5 h (preferably 3 h), followed by centrifugation at 3000-8000 r/min (preferably 5000 r/min) for 5-30 min (preferably 20 min), and finally the supernatant was removed and the precipitate was taken out for storage at 2-8° C. The working solution was diluted 100-5000 times (preferably 100 times) with PBS buffer for later use. The labeling effect was better and more stable with the preferred solution.

2. Biotin-Labeled a Mouse Anti-Human HE4 Monoclonal Antibody:

A mouse anti-human HE4 monoclonal antibody was diluted into 1 mg/mL with sodium carbonate buffer solution 2, and dialyzed against sodium carbonate buffer solution 2 after stirring for 1-5 h (preferably 4 h) at room temperature (25° C.±5° C.) in the dark; then 6-aminocaproic acid-N-hydroxysuccinimide-biotin (BCNHS) was prepared into 1 mg/mL solution by using N,N-dimethylamide (DMF); 10-160 μL (preferably 80 μL) of the above DMF solution was added to 1 mL of a mouse anti-human HE4 monoclonal antibody solution, the mixture was blended in a glass bottle, and stirred for 1-5 h (preferably 2 h) at room temperature (25° C.±5° C.) in the dark; 5-20 μL (preferably 9.6 μL) of 1 mol/L ammonium chloride solution was added to the above mixture, and stirred for 10 min at room temperature (25° C.±5° C.) in the dark; the mixed solution was then transferred to a dialysis bag and dialyzed against a phosphate buffer at 4° C. overnight. Finally, The dialyzed substance was taken out and stored at −20° C. by adding the same amount of glycerol. The working solution was diluted 100-5000 times (preferably 300 times) with PBS buffer for later use. The labeling effect was better and more stable with the preferred solution.

3. Another Magnetic Microspheres-Labeled Mouse Anti-Human HE4 Monoclonal Antibody:

0.5 mL of 2.5 mg/mL magnetic microspheres (with a particle size of 300 nm-3000 nm, preferably 800 nm, purchased from Sigma) were washed twice with a phosphate buffer solution (pH=7.4) and then re-suspended in 10 mL of the phosphate buffer solution, 4 mL of glutaraldehyde was sequentially added for shaking reaction for 10 h at room temperature, the reactant was washed for 2-3 times with purified water after finishing the reaction, and the magnetic microspheres were re-suspended in 10 mL of the phosphate buffer solution; another mouse anti-human HE4 monoclonal antibody was diluted into 1 mg/mL with sodium carbonate buffer solution 2, and dialyzed against sodium carbonate buffer solution 2 after stirring for 1-5 h (preferably 2 h) at room temperature (25° C.±5° C.) in the dark; 0.5-5 mL (preferably 1 mL) another mouse anti-human HE4 monoclonal antibody was pipetted and added into 10 mL of phosphate buffer solution of the magnetic microspheres for shaking for 8 h at room temperature; the magnetic microspheres were sucked on one side by using a magnet, washed 3 times by using PBS, and then dispersed in 10 mL of phosphate buffer solution. The labeling effect was better and more stable with the preferred solution.

4. The Detection Process was as Follows:

(1) 10-100 μL (preferably 25 μL) of HE4-containing sample to be tested, 10-150 μL (preferably 100 μL) of biotin-labeled mouse anti-human HE4 monoclonal antibody and 10-150 μL (preferably 50 μL) of another magnetic microspheres-labeled mouse anti-human HE4 monoclonal antibody were added into a reaction cell and incubated for 3-90 min (preferably 15 min) to form a HE4 immune complex labeled with magnetic microspheres at one end and with biotin at the other end;

(2) 10-100 μL (preferably 50 μL) of nano copper oxide with surface-labeled streptavidin was added into the mixture reacted from step (1), and the biotin labeled at one end of the HE4 immune complex specifically bound to the streptavidin labeled on the surface of the nano copper oxide, to form the magnetic microspheres binding the HE4 immune complex labeled with nano copper oxide at one end;

(3) the magnetic microspheres with surface-bound the HE4 immune complex in step (2) were fixed to the surface of the working electrode in a magnetic separation method, the residual liquid in the reaction cell was removed, and 25-1000 μL (preferably 100 μL) of citric acid electrolyte was filled;

(4) a four-electrode system was used for the measurement; the electrochemical detection was performed on screen printing electrodes of a working electrode (graphite electrode), an internal control electrode (glassy carbon electrode), a counter electrode (platinum electrode or carbon electrode) and a reference electrode (Ag/AgCl electrode). Firstly, the internal control electrode, the counter electrode and the reference electrode were connected to the electrochemical workstation, a voltammogram of the substrate was measured, the cardinality calibration was performed, and then the working electrode, the counter electrode and the reference electrode were connected to the electrochemical workstation, and a voltammogram of reduction reaction of nano copper oxide on the immune complex on the surface of the magnetic microspheres was measured by utilizing a voltammetry method; and then characteristic peak of copper ions in the measured voltammogram was picked out, a half-peak area was calculated, a curve was fitted between the half-peak area and the concentration of the HE4 using a Log-Log linear regression equation or a four-parameter equation (preferably a four-parameter equation) to obtain HE4 standard curve, and the content of the HE4 was calculated by the standard curve.

And the detection sensitivity was higher and the stability was better by adopting the preferred solution in each step.

5. Establishment of a Standard Curve:

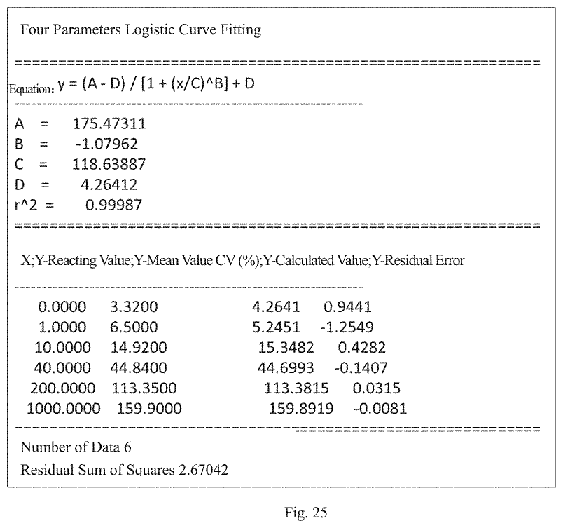

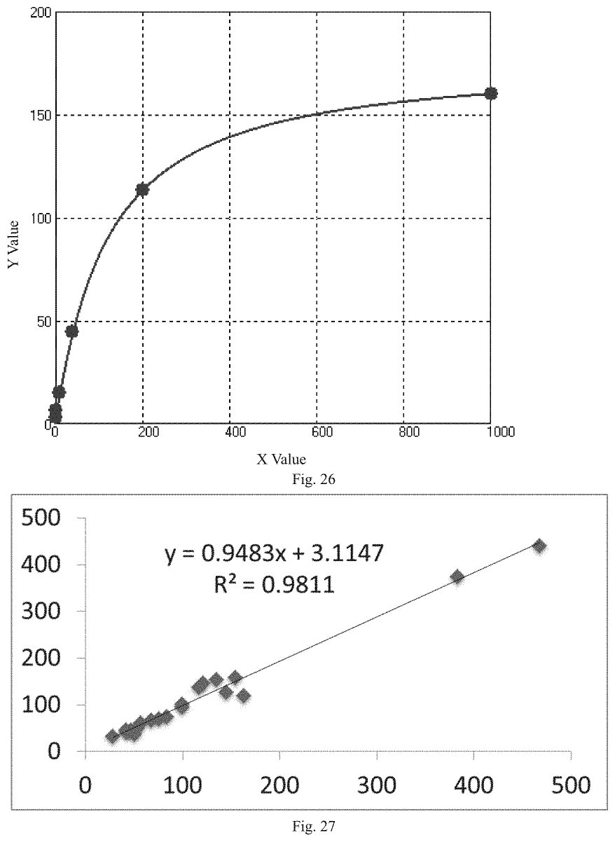

HE4 calibrators at concentrations of 0, 1, 10, 40, 200, 1000 pmol/L were used to establish a HE4 standard curve, with a detection sensitivity of 0.2 pmol/L, and a detection range of 1-1000 pmol/L; the detection data are shown in Table 12, the four-parameter Logistic curve fitting data are shown in FIG. 25, and the standard curve is shown in FIG. 26.

TABLE 12
Detection Data

| Standard concentration | |
|---|---|
| (pmol/L) | Integral area |
| 0 | 3.32 |
| 1 | 6.50 |
| 10 | 14.92 |
| 40 | 44.84 |
| 200 | 113.35 |
| 1000 | 159.90 |

6. Comparison with Roche's Results:

20 samples were detected by adopting the method of the present invention and Roche's electrochemiluminescence method; the detection results are shown in Table 13, the correlation analysis of the detection results is shown in FIG. 27, the regression equation is y=0.9483x+3.1147, and R²=0.9811, indicating that the method has good correlation with the Roche's electrochemiluminescence method.

TABLE 13
Comparison of the results of this method with
that of the Roche's electrochemical method

| This method (pmol/L) | Roche's (pmol/L) |
|---|---|
| 154.36 | 157.64 |
| 42.50 | 41.55 |
| 75.95 | 69.48 |
| 467.59 | 440.03 |
| 28.57 | 32.73 |
| 121.29 | 146.08 |
| 116.62 | 136.62 |
| 83.93 | 73.26 |
| 99.53 | 101.14 |
| 382.86 | 374.4 |
| 99.40 | 94.73 |
| 53.53 | 49.43 |
| 50.69 | 35.91 |
| 46.98 | 46.09 |
| 67.76 | 66.21 |
| 145.23 | 126.08 |
| 41.59 | 45.24 |
| 134.91 | 153.69 |
| 163.10 | 118.92 |
| 56.78 | 60.52 |

### Embodiment 9: Taking 25-Hydroxyvitamin D (25-OH-D) as an Example (a Competition Method)

1. Nano Copper Oxide-Labeled Streptavidin:

Streptavidin was diluted into 1 mg/mL with a PBS buffer solution, and dialyzed against PBS buffer solution after stirring for 4 h at room temperature (25° C.±5° C.) in the dark for later use; 1 mL of streptavidin solution was added to 1-10 mL (preferably 3 mL) of prepared nano copper oxide (1-100 nm, preferably 30-40 nm) material (mass fraction 1 g/100 mL, purity 99.9%), the mixture was magnetically stirred for 30 min at 25° C.±5° C. in the dark; the supernatant was then removed by centrifugation at 10,000 r/min for 5-30 min (preferably 15 min), and the precipitate was re-dispersed and blocked with 10 mL of 1-10% (preferably 10%) BSA for 1-5 h (preferably 3 h), followed by centrifugation at 3000-8000 r/min (preferably 5000 r/min) for 5-30 min (preferably 20 min), and finally the supernatant was removed and the precipitate was taken out for storage at 2-8° C. The working solution was diluted 100-5000 times (preferably 100 times) with PBS buffer for later use. The labeling effect was better and more stable with the preferred solution.

2. A Biotin-Labeled 25-OH-D Complete Antigen:

A 25-OH-D complete antigen was diluted into 1 mg/mL with sodium carbonate buffer solution 2, and dialyzed against sodium carbonate buffer solution 2 after stirring for 1-5 h (preferably 4 h) at room temperature (25° C.±5° C.) in the dark; then 6-aminocaproic acid-N-hydroxysuccinimide-biotin (BCNHS) was prepared into 1 mg/mL solution by using N,N-dimethylamide (DMF); 10-160 μL (preferably 80 μL) of the above DMF solution was added to 1 mL of 25-OH-D complete antigen solution, the mixture was blended in a glass bottle, and stirred for 1-5 h (preferably 2 h) at room temperature (25° C.±5° C.) in the dark; 5-20 μL (preferably 9.6 μL) of 1 mol/L ammonium chloride solution was added to the above mixture, and stirred for 10 min at room temperature (25° C.±5° C.) in the dark; the mixed solution was then transferred to a dialysis bag and dialyzed against a phosphate buffer at 4° C. overnight. Finally, The dialyzed substance was taken out and stored at −20° C. by adding the same amount of glycerol. The working solution was diluted 100-5000 times (preferably 300 times) with a PBS buffer for later use. The labeling effect was better and more stable with the preferred solution.

3. A Magnetic Microspheres-Labeled Mouse Anti-Human 25-OH-D Monoclonal Antibody:

0.5 mL of 2.5 mg/mL magnetic microspheres (particle size of 300 nm-3000 nm, preferably 800 nm, purchased from Sigma) were washed twice with a phosphate buffer solution (pH=7.4) and then re-suspended in 10 mL of the phosphate buffer solution, 4 mL of glutaraldehyde was sequentially added for shaking reaction for 10 h at room temperature, the reactant was washed for 2-3 times with purified water after finishing the reaction, and the magnetic microspheres were re-suspended in 10 mL of the phosphate buffer solution; a mouse anti-human 25-OH-D monoclonal antibody was diluted into 1 mg/mL with sodium carbonate buffer solution 2, and dialyzed against sodium carbonate buffer solution 2 after stirring for 1-5 h (preferably 2 h) at room temperature (25° C.±5° C.) in the dark; 0.5-5 mL (preferably 1 mL) a mouse anti-human 25-OH-D monoclonal antibody was pipetted and added into 10 mL of phosphate buffer solution of the magnetic microspheres for shaking for 8 h at room temperature; the magnetic microspheres were sucked on one side by using a magnet, washed 3 times by using PBS, and then dispersed in 10 mL of phosphate buffer solution. The labeling effect was better and more stable with the preferred solution.

4. The Detection Process was as Follows:

(1) 10-100 μL (preferably 25 μL) of 25-OH-D-containing sample to be tested was treated with 25 μL of binding protein releasing agent for 10 min, then added into a reaction cell together with 10-150 μL (preferably 100 μL) of biotin-labeled 25-OH-D complete antigen and 10-150 μL (preferably 50 μL) of magnetic microspheres-labeled mouse anti-human 25-OH-D monoclonal antibody, the mixture was incubated for 3-90 min (preferably 15 min), wherein the 25-OH-D in the sample to be tested competed with the biotin-labeled 25-OH-D complete antigen for an immunoreaction with the mouse anti-human 25-OH-D monoclonal antibody, to form the magnetic microspheres with surface-bound the immune complex I with 25-OH-D in the sample to be tested and immune complex II with a biotin-labeled 25-OH-D complete antigen;

(2) 10-100 μL (preferably 50 μL) of nano copper oxide with surface-labeled streptavidin was added into the mixture reacted from step (1), and the biotin labeled at one end of the 25-OH-D immune complex specifically bound to the streptavidin labeled on the surface of the nano copper oxide, to form the magnetic microsphere with surface-bound 25-OH-D immune complex;

(3) the magnetic microspheres with surface-bound immune complex in step (2) were fixed to the surface of the working electrode in a magnetic separation method, the residual liquid in the reaction cell was removed, and 25-1000 μL (preferably 100 μL) citric acid electrolyte was filled;

(4) a four-electrode system was used for the measurement; the electrochemical detection was performed on screen printing electrodes of a working electrode (lead electrode), an internal control electrode (gold microelectrode), a counter electrode (platinum electrode or carbon electrode) and a reference electrode (Ag/AgCl electrode). Firstly, the internal control electrode, the counter electrode and the reference electrode were connected to the electrochemical workstation, the voltammetric curve of the substrate was determined for cardinality calibration, the working electrode, counter electrode, and reference electrode were connected to the electrochemical workstation, and a voltammogram of reduction reaction of nano copper oxide on the immune complex on the surface of the magnetic microspheres was measured by utilizing a voltammetry method; and then the characteristic peak of copper ions in the measured voltammogram was picked out, a half-peak area was calculated, a curve was fitted between the half-peak area and the concentration of the 25-OH-D using a Log-Logit linear regression equation or a four-parameter equation (preferably a four-parameter equation) to obtain 25-OH-D standard curve, and the content of the 25-OH-D was calculated by the standard curve.

And the detection sensitivity was higher and the stability was better by adopting the preferred solution in each step.

5. Establishment of a Standard Curve:

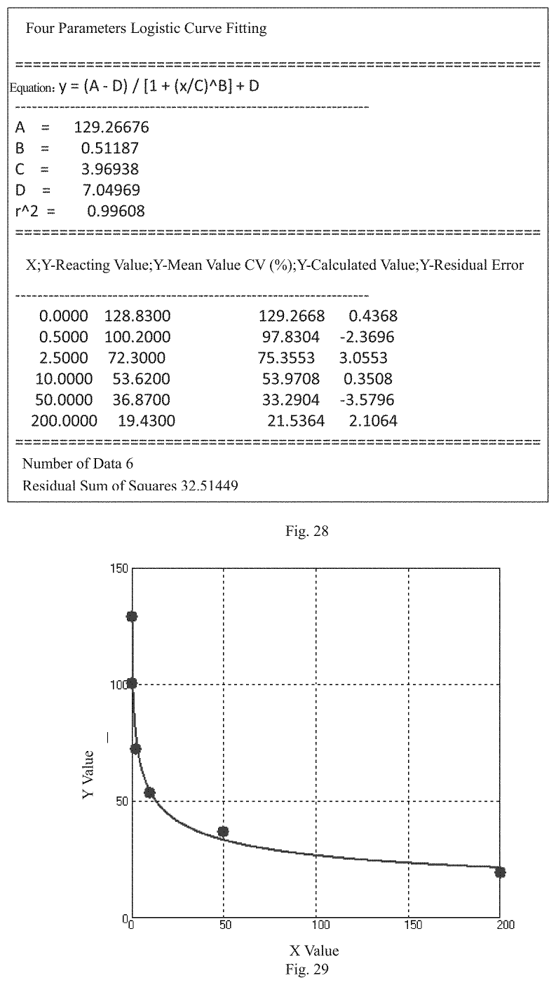

A 25-OH-D calibrators at concentrations of 0, 0.5, 2.5, 10, 50, 200 ng/mL were used to establish a 25-OH-D standard curve, with a detection sensitivity of 0.2 ng/mL and a detection range of 0.5-200 ng/mL; the detection data are shown in Table 14, the four-parameter Logistic curve fitting data are shown in FIG. 28 and the standard curve is shown in FIG. 29.

TABLE 14
Detection Data

| Standard concentration | |
|---|---|
| (ng/mL) | Integral area |
| 0 | 128.83 |
| 0.5 | 100.2 |
| 2.5 | 72.32 |
| 10 | 53.62 |
| 50 | 36.87 |
| 200 | 19.43 |

6. Comparison with Roche's Results:

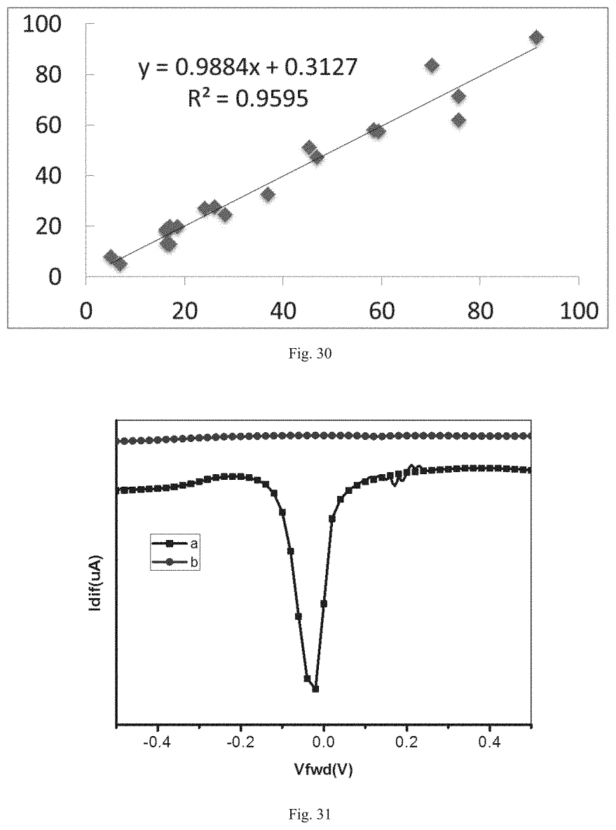

20 samples were detected by adopting the method of the present invention and Roche's electrochemiluminescence method; the detection results are shown in Table 15, the correlation analysis of the detection results is shown in FIG. 30, the regression equation is y=0.9884x+0.3127, and R²=0.9595, indicating the method has good correlation with the Roche's electrochemiluminescence method.

TABLE 15
Comparison of the results of this method with
that of the Roche's electrochemical method

| This method (ng/mL) | Roche's (ng/mL) |
|---|---|
| 75.61 | 71.16 |
| 18.67 | 19.58 |
| 46.99 | 47.19 |
| 17.06 | 19.8 |
| 45.37 | 51.14 |
| 16.55 | 13.1 |
| 5.12 | 7.7 |
| 16.31 | 18.16 |
| 36.94 | 32.38 |
| 58.41 | 57.95 |
| 70.23 | 83.35 |
| 24.23 | 26.81 |
| 17.11 | 12.9 |
| 6.95 | 5.09 |
| 75.73 | 61.88 |
| 26.22 | 27.58 |
| 16.33 | 18.51 |
| 59.49 | 57.32 |
| 28.34 | 24.45 |
| 91.48 | 94.6 |

### Embodiment 10: Comparison of the Stability of CuO and Cu²⁺ Loaded on Polystyrene Microsphere as Labeling Materials

(1) Cu²⁺ was loaded on polystyrene (PS) microsphere to serve as a material to label a mouse anti-human PCT monoclonal antibody, then the labeled antibody was used for measuring a 5 ng/mL PCT calibrator, the measured voltammogram of the reduction reaction of Cu²⁺ was shown as line a in FIG. 31, and a higher reduction peak signal was generated near 0V; then, a mouse anti-human PCT monoclonal antibody labeled with Cu²⁺ loaded on PS microsphere was placed under 37° C. for 3 days, and then used for measuring a 5 ng/mL PCT calibrator, the measured voltammogram of reduction reaction of Cu²⁺ was shown as line b in FIG. 31, and little peak of the reduction reaction of Cu²⁺ was found, indicating that the stability of Cu²⁺ loaded on polystyrene (PS) microsphere as a labeling material is poor, which may be caused by the release of Cu²⁺ from PS microsphere with the extension of the holding time.

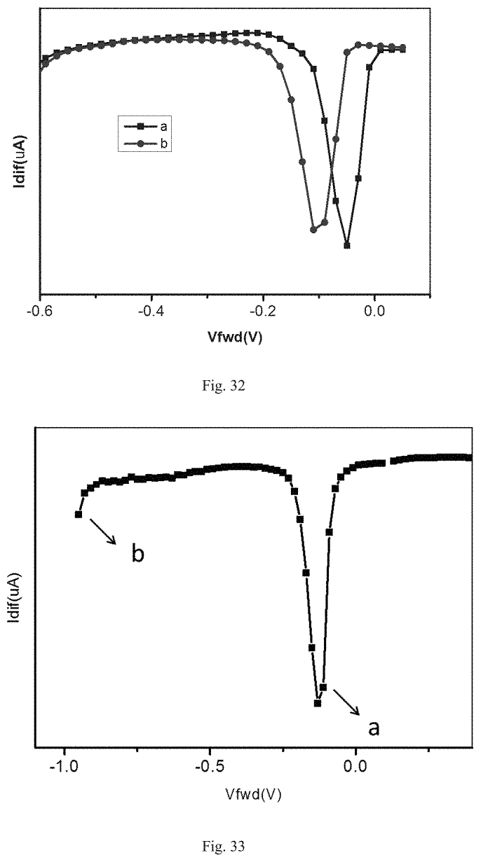

(2) CuO as a labeling material was used to label a mouse anti-human PCT monoclonal antibody, then the labeled antibody was used for measuring a 5 ng/mL PCT calibrator, and the measured voltammogram of the reduction reaction of the CuO was shown as line a in FIG. 32; then a CuO-labeled mouse anti-human PCT monoclonal antibody was placed under 37° C. for 3 days and then was used for measuring a 5 ng/mL PCT calibrator; the measured voltammogram of reduction reaction of the CuO was shown as line b in FIG. 32; it can be seen from the FIG. 26 that the detection results are similar, indicating that CuO has good stability as a tracer; in addition, Cu²⁺ is present in an ionic state in the solution, which needs to be loaded on a carrier (for example, PS microsphere, platinum microsphere, SiO₂ microsphere) as a labeling material, and then labeled on the antibody or antigen to be tested, so that the process is complicated; while CuO is present in solution as a small nanoparticle, which can be directly used for labeling of the antibody or antigen of the analyte, so that the operation process is simplified.

### Embodiment 11: Comparison of Cu²⁺ Reduction Peak in Copper Oxide and Zn²⁺ Reduction Peak in Zinc Oxide Affected by H⁺ Reduction Peak

(1) CuO as a labeling material was used to label a mouse anti-human PCT monoclonal antibody, then the labeled antibody was used for measuring a 25 ng/mL PCT calibrator; the measured voltammogram of reduction reaction of the CuO is shown in FIG. 33, wherein a is the reduction peak of Cu²⁺ in CuO, which is hardly affected by the H⁺ reduction peak which may exist at b.

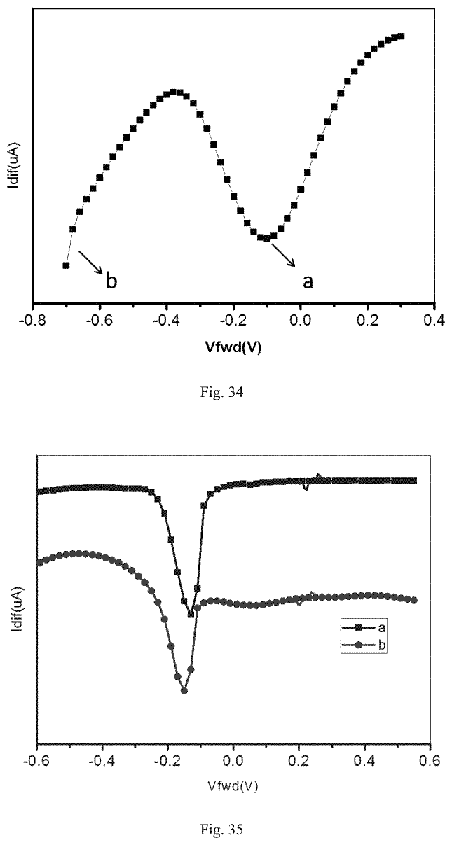

(2) ZnO as a labeling material was used to label a mouse anti-human PCT monoclonal antibody, then the labeled antibody was used for measuring a 25 ng/mL PCT calibrator; the measured voltammogram of reduction reaction of the ZnO is shown in FIG. 34, wherein a is the reduction peak of Zn²⁺ in ZnO, which is greatly affected by the H⁺ reduction peak which may exist at b, which has a great influence on the detection results.

### Embodiment 12: Effects of the Internal Control Electrode

CuO as a labeling material was used to label a mouse anti-human PCT monoclonal antibody, then the labeled antibody was used for measuring a 5 ng/mL PCT calibrator. The internal control electrode was used to perform the cardinal calibration, and then the voltammogram of reduction reaction of CuO as a labeling material was measured, as shown as line a in FIG. 35, by deducting the background interference, the peak shape was regular with a smaller error and improved accuracy of detection; the voltammogram of reduction reaction of CuO as a labeling material was directly measured without using the internal control electrode for cardinal calibration, as shown as line b in FIG. 35, the reduction peak of copper ions in CuO was affected by the background, and the peak shape was not regular, which may cause a larger error in the detection results.

Attachment: Preparation of the required solution

| (1) | PB buffer solution | | |
|---|---|---|---|
| | Sodium dihydrogen phosphate | 0.99 | g |
| | Disodium hydrogen phosphate | 5.16 | g |
| | Tween-20 | 1 | mL |
| | Proclin300 | 1 | mL |
| | Filling up the volume to 1000 mL with purified water; | | |
| (2) | Sodium carbonate buffer solution 1; | | |
| | Sodium carbonate | 0.99 | g |
| | Sodium hydrogen carbonate | 2.96 | g |
| | Filling up the volume to 1000 mL with purified water, | | |
| (3) | Phosphate buffer solution | | |
| | Sodium dihydrogen phosphate | 0.99 | g |
| | Disodium hydrogen phosphate | 5.16 | g |
| | Filling up the volume to 1000 mL with purified water; | | |
| (4) | PBS buffer solution | | |
| | Sodium dihydrogen phosphate | 0.99 | g |
| | Disodium hydrogen phosphate | 5.16 | g |
| | NaCl | 9.00 | g |
| | Filling up the volume to 1000 mL with purified water; | | |
| (5) | Sodium carbonate buffer solution 2 | | |
| | Sodium carbonate | 4.33 | g |
| | Sodium hydrogen carbonate | 2.96 | g |
| | Filling up the volume to 1000 mL with purified water; | | |
| (6) | Citric acid electrolyte | | |
| | Trisodium citrate | 7.33 | g |
| | Citric acid | 4.44 | g |
| | Sodium hydroxide | 1 | g |

Filling up the volume to 1000 mL with purified water.

While embodiments of the present invention have been described in detail above, the foregoing is only a preferred embodiment of the invention and is not to be considered as limiting the scope of the invention. All changes and modifications that come within the meaning and range of equivalency of the claims are to be embraced therein.

What is claimed is:

1. A method of electrochemical detection based on tracers labeling, comprising the following steps:
(1) preparing a tracer-labeled immune complex in a reaction cell;
(2) enriching the tracer-labeled immune complex on a surface of a working electrode through a separation method, removing residual liquid in the reaction cell, and filling the reaction cell with an electrolyte;
(3) connecting the working electrode to an electrochemical workstation, measuring a voltammogram of a tracer of the tracer-labeled immune complex by a voltammetric method to obtain a measured voltammogram, picking out a characteristic peak of the tracer in the measured voltammogram, calculating a half-peak area, fitting a curve between the half-peak area and a concentration of an analyte by using a regression equation to obtain a standard curve, and calculating a content of the analyte by the standard curve.

2. The method of the electrochemical detection based on the tracers labeling of claim 1, wherein in step (3), the characteristic peak is located within a range from −100 mV to +100 mV of a theoretical characteristic peak of metal ions; the regression equation is a Log-Log regression equation or a Log-Logit regression equation.

3. The method of the electrochemical detection based on the tracers labeling of claim 2, wherein the tracer-labeled immune complex is prepared by the following steps:
(a), labeling a first antibody of the analyte with the tracer to obtain a tracer-labeled antibody of the analyte;
(b), labeling a second antibody of the analyte with one of a pair of substances having specific affinity to obtain a substance-labeled antibody;
(c), labeling a nano-microsphere with an other of the pair of substances having the specific affinity; and
(d), adding a sample containing the analyte, the tracer-labeled antibody of the analyte, and the substance-labeled antibody into the reaction cell, performing an incubation reaction on the reaction cell for 3-90 min, and sequentially adding the nano-microsphere with surface-labeled the other of the pair of substances having the specific affinity to form the tracer-labeled immune complex;
or,
(a), labeling an antibody of the analyte with the tracer to obtain a tracer-labeled antibody of the analyte;
(b), labeling a complete antigen of the analyte with one of a pair of substances having specific affinity to obtain a substance-labeled complete antigen;
(c), labeling a nano-microsphere with an other of the pair of substances having the specific affinity; and
(d), adding a sample containing the analyte, the tracer-labeled antibody of the analyte, and the substance-labeled complete antigen into the reaction cell, performing an incubation reaction on the reaction cell for 3-90 min, and sequentially adding the nano-microsphere with surface-labeled the other of the pair of substances having the specific affinity to form the tracer-labeled immune complex;
or,
(a), labeling a complete antigen of the analyte with a tracer to obtain a tracer-labeled complete antigen of the analyte;
(b), labeling an antibody of the analyte with one of a pair of substances having specific affinity to obtain a substance-labeled antibody;
(c), labeling a nano-microsphere with an other of the pair of substances having the specific affinity; and
(d), adding a sample containing the analyte, the tracer-labeled complete antigen of the analyte, and the substance-labeled antibody into the reaction cell, performing an incubation reaction on the reaction cell for 3-90 min, and sequentially adding the nano-microsphere with surface-labeled the other of the pair of substances having the specific affinity to form the tracer-labeled immune complex.

4. The method of the electrochemical detection based on the tracers labeling of claim 3, wherein the tracer is a metal ion material; the electrochemical detection is performed by a three-electrode system or a two-electrode system.

5. The method of the electrochemical detection based on the tracers labeling of claim 4, wherein the metal ion material is a microsphere containing a metal ion on a surface or inside of the microsphere; the metal ion is one selected from the group consisting of Cd²⁺, Cu²⁺, Zn²⁺, Mn²⁺, Pb²⁺, Ag⁺, Li⁺, Hg²⁺, Co²⁺, Cr³⁺, Ni²⁺, Au³⁺, and Ba²⁺ ions; the microsphere is one selected from the group consisting of polystyrene microsphere, polytetrafluoroethylene microsphere, titanium dioxide microsphere, manganese dioxide microsphere, zirconium dioxide microsphere, organic silicon microsphere, polyamide microsphere, polyacrylic acid microsphere, chitosan microsphere, polyaniline microsphere, polyvinyl chloride microsphere, cobalt microsphere, nickel microsphere, platinum microsphere, gold microsphere, silver microsphere, palladium microsphere, silicon dioxide microsphere and magnetic microsphere.

6. The method of the electrochemical detection based on the tracers labeling of claim 5, wherein the microsphere is one selected from the group consisting of polystyrene microsphere, polytetrafluoroethylene microsphere, silica microsphere, titanium dioxide microsphere, organic silicon microsphere, polyamide microsphere, polyacrylic acid microsphere, chitosan microsphere, polyaniline microsphere, polyvinyl chloride microsphere, and magnetic microsphere.

7. The method of the electrochemical detection based on the tracers labeling of claim 6, wherein the separation method is performed by a centrifugal separation, an electric field action, or a capillary action; or when the microsphere is made of magnetic microsphere, the separation method is performed by a magnetic separation; the magnetic microsphere is a magnetic Fe₃O₄ microsphere, a magnetic γ-Fe₂O₃ microsphere, a magnetic Pt microsphere, a magnetic Ni microsphere, or a magnetic Co microsphere, or a core/shell or doped microsphere formed by combining magnetic Fe₃O₄, γ-Fe₂O₃, Pt, Ni or Co with an inorganic matter or an organic matter.

8. The method of the electrochemical detection based on the tracers labeling of claim 7, wherein the metal ion material has a particle size of 1-500 nm and the microsphere has a particle size of 50 nm-5 μm.

9. The method of the electrochemical detection based on the tracers labeling of claim 1, wherein in step (3), the regression equation is a four-parameter regression equation.

10. The method of the electrochemical detection based on the tracers labeling of claim 9, wherein the tracer-labeled immune complex is prepared by the following steps:
(a), labeling a first antibody or an antigen of the analyte with the tracer to obtain a tracer-labeled antibody of a tracer-labeled antigen;
(b), labeling second antibody of the analyte with one of a pair of substances having specific affinity to obtain a substance-labeled antibody;
(c), labeling a magnetic microsphere with an other of the pair of substances having with specific affinity; and
(d), adding the analyte, the tracer-labeled antibody or the tracer-labeled antigen, and the the substance-labeled antibody into a detection cell, performing an incubation reaction on the detection cell, and sequentially adding the magnetic microsphere labeled with the other of the pair of substances having the specific affinity to form the tracer-labeled immune complex;
or,
(a), labeling a first antibody or an antigen of the analyte with a tracer to obtain a tracer-labeled antibody or a tracer-labeled antigen;
(b), labeling a second antibody of the analyte with a magnetic microsphere to obtain a magnetic microsphere-labeled antibody of the analyte; and
(c), adding the analyte, the tracer-labeled antibody or the tracer-labeled antigen of the analyte, and the magnetic microsphere-labeled antibody of the analyte into a detection cell, and performing an incubation reaction on the detection cell to form the tracer-labeled immune complex;
or,
(a), labeling a first antibody or an antigen of the analyte with one of a pair of substances having specific affinity to obtain a substance-labeled antibody or a substance-labeled antigen;
(b), labeling a second antibody of the analyte with a magnetic microsphere to obtain a magnetic microsphere-labeled antibody;
(c), labeling an another of the pair of substances having the specific affinity with the tracer; and
(d), adding the analyte, the substance-labeled antibody or the substance-labeled antigen, and the magnetic microsphere-labeled antibody of the analyte into a detection cell, performing an incubation reaction on the detection cell, and sequentially adding the other of the pair of substances having the specific affinity labeled with the tracer to form the tracer-labeled immune complex.

11. The method of the electrochemical detection based on the tracers labeling of claim 10, wherein the tracer-labeled immune complex comprises the tracer-labeled antibody or the tracer-labeled antigen, the magnetic microsphere-labeled antibody of the analyte, and the analyte.

12. The method of the electrochemical detection based on the tracers labeling of claim 11, wherein the tracer-labeled antibody or the tracer-labeled antigen is linked by the pair of substances having the specific affinity.

13. The method of the electrochemical detection based on the tracers labeling of to claim 12, wherein the pair of substances having the specific affinity is a pair formed by biotin and streptavidin, a pair formed by biotin and avidin, a pair formed by fluorescein and anti-fluorescein, or a pair formed by a third antibody and a secondary antibody specifically binding the third antibody.

14. The method of the electrochemical detection based on the tracers labeling of claim 13, wherein the magnetic microsphere-labeled antibody of the analyte is linked by the pair of substances having the specific affinity.

15. The method of the electrochemical detection based on the tracers labeling of claim 14, wherein the pair of substances having specific affinity is a pair formed by biotin and streptavidin, a pair formed by biotin and avidin, a pair formed by fluorescein and anti-fluorescein, or a pair formed by a fourth antibody and a secondary antibody specifically binding the fourth antibody.

16. The method of the electrochemical detection based on the tracers labeling of claim 15, wherein the tracer is a metal oxide material; the electrochemical detection is performed by a four electrode system.

17. The method of the electrochemical detection based on the tracers labeling of claim 16, wherein the metal oxide material is copper oxide.

18. The method of the electrochemical detection based on the tracers labeling of claim 17, wherein the copper oxide is one selected from the group consisting of a bare copper oxide nanoparticle, a copper oxide with a surface coated by a layer of one selected from the group consisting of silicon dioxide, titanium dioxide, carbonate, silicate, phosphate, silicon carbide, graphite and silicon nitride, and a copper oxide with a surface coated by a layer of one selected from the group consisting of organic silicon, polystyrene, polytetrafluoroethylene, polyamide, polyethylene, polyvinyl chloride, polyvinyl fluoride, polyacrylonitrile, polyamide, polyimide, polyaniline, polypyrrole, polyacrylic acid, chitosan, polylactic acid, epoxy resin, phenolic resin, polyacetylene, polyester, β-cyclodextrin polymer, vitamin, and melamine.

19. The method of the electrochemical detection based on the tracers labeling of claim 16, wherein the four-electrode system comprises a screen printing electrode consisting of a working electrode, an internal control electrode, a counter electrode and a reference electrode respectively, the screen printing electrode is inserted into the detection cell, and a magnet is arranged under the working electrode of the screen printing electrode in the detection cell.

20. The method of the electrochemical detection based on the tracers labeling of claim 19, wherein the working electrode is:
one selected from the group consisting of a copper electrode, a carbon electrode, a glassy carbon electrode, a gold microelectrode, a graphite electrode, a silver electrode, and a lead electrode, or
an electrode doped with graphene or fullerene, wherein the electrode is one selected from the group consisting of a copper electrode, a carbon electrode, a glassy carbon electrode, a gold microelectrode, a graphite electrode, a silver electrode, and a lead electrode, or
an electrode modified, coated, doped or attached to graphene or fullerene on a surface of the electrode, wherein the electrode is one selected from the group consisting of a copper electrode, a carbon electrode, a glassy carbon electrode, a gold microelectrode, a graphite electrode, a silver electrode, and a lead electrode;
the internal control electrode is:
one selected from the group consisting of a copper electrode, a carbon electrode, a glassy carbon electrode, a gold microelectrode, a graphite electrode, a silver electrode, and a lead electrode, or
an electrode doped with graphene or fullerene, wherein the electrode is one selected from the group consisting of a copper electrode, a carbon electrode, a glassy carbon electrode, a gold microelectrode, a graphite electrode, a silver electrode, and a lead electrode, or
an electrode modified, coated, doped or attached to graphene or fullerene on a surface of the electrode, wherein the electrode is one selected from the grout consisting of a copper electrode, a carbon electrode, a glassy carbon electrode, a gold microelectrode, a graphite electrode, a silver electrode, and a lead electrode;
the counter electrode is a platinum wire electrode or a carbon electrode; and
the reference electrode is a calomel electrode or an Ag/AgCl electrode.

21-30. (canceled)
